SEQUENCE LISTING (65 sequences)

SEQ ID NO: 1 - DNA, 53 bp, Artificial Sequence
  Description of Artificial Sequence: Synthetic oligonucleotide
  modified_base (48)..(53): a, c, t, g, unknown or other
gaaattaatacgactcactatagggaagacgtgtgctcttccgatctnnnnnn

SEQ ID NO: 2 - DNA, 56 bp, Artificial Sequence
  Description of Artificial Sequence: Synthetic oligonucleotide
  modified_base (48)..(56): a, c, t, g, unknown or other
gaaattaatacgactcactatagggaagacgtgtgctcttccgatctnnnnnnnnn

SEQ ID NO: 3 - DNA, 62 bp, Artificial Sequence
  Description of Artificial Sequence: Synthetic oligonucleotide
  modified_base (48)..(62): a, c, t, g, unknown or other
gaaattaatacgactcactatagggaagacgtgtgctcttccgatctnnnnnnnnnnnnnnn

SEQ ID NO: 4 - DNA, 47 bp, Artificial Sequence
  Description of Artificial Sequence: Synthetic oligonucleotide
gaaattaatacgactcactatagggaagacgtgtgctcttccgatct

SEQ ID NO: 5 - DNA, 37 bp, Artificial Sequence
  Description of Artificial Sequence: Synthetic oligonucleotide
ggatctcgacgctctccctcaaccctgcttgcaggat

SEQ ID NO: 6 - DNA, 41 bp, Artificial Sequence
  Description of Artificial Sequence: Synthetic oligonucleotide
ggatctcgacgctctccctcctccatctctttgtcggtggt

SEQ ID NO: 7 - DNA, 38 bp, Artificial Sequence
  Description of Artificial Sequence: Synthetic oligonucleotide
ggatctcgacgctctccctatgaggaaggcccctgtgc

SEQ ID NO: 8 - DNA, 41 bp, Artificial Sequence
  Description of Artificial Sequence: Synthetic oligonucleotide
ggatctcgacgctctccctccccagagttcatggatgcact

SEQ ID NO: 9 - DNA, 38 bp, Artificial Sequence
  Description of Artificial Sequence: Synthetic oligonucleotide
ggatctcgacgctctccctaaagcagccctctcccagg

SEQ ID NO: 10 - DNA, 43 bp, Artificial Sequence
  Description of Artificial Sequence: Synthetic oligonucleotide
ggatctcgacgctctccctatttctgagatcaggtctgacaag

SEQ ID NO: 11 - DNA, 38 bp, Artificial Sequence
  Description of Artificial Sequence: Synthetic oligonucleotide
ggatctcgacgctctcccttgctgaaggagggtcaccg

SEQ ID NO: 12 - DNA, 40 bp, Artificial Sequence
  Description of Artificial Sequence: Synthetic oligonucleotide
ggatctcgacgctctccctttcaagcagctggtggaagac

SEQ ID NO: 13 - DNA, 39 bp, Artificial Sequence
  Description of Artificial Sequence: Synthetic oligonucleotide
ggatctcgacgctctccctcagcccagcttgccaatggc

SEQ ID NO: 14 - DNA, 37 bp, Artificial Sequence
  Description of Artificial Sequence: Synthetic oligonucleotide
ggatctcgacgctctccctgtctggttggccggcagc

SEQ ID NO: 15 - DNA, 42 bp, Artificial Sequence
  Description of Artificial Sequence: Synthetic oligonucleotide
ggatctcgacgctctccctgacgttgtgcaaggagagaacct

SEQ ID NO: 16 - DNA, 37 bp, Artificial Sequence
  Description of Artificial Sequence: Synthetic oligonucleotide
ggatctcgacgctctccctcgcctcgtcagcctccac

SEQ ID NO: 17 - DNA, 39 bp, Artificial Sequence
  Description of Artificial Sequence: Synthetic oligonucleotide
ggatctcgacgctctccctaccagtggtgtgttggagct

SEQ ID NO: 18 - DNA, 37 bp, Artificial Sequence
  Description of Artificial Sequence: Synthetic oligonucleotide
ggatctcgacgctctccctcgaagcagccctccccaa

SEQ ID NO: 19 - DNA, 37 bp, Artificial Sequence
  Description of Artificial Sequence: Synthetic oligonucleotide
ggatctcgacgctctccctaccaggtccgacaggtcc

SEQ ID NO: 20 - DNA, 37 bp, Artificial Sequence
  Description of Artificial Sequence: Synthetic oligonucleotide
ggatctcgacgctctccctcatggacaagcccgccaa

SEQ ID NO: 21 - DNA, 38 bp, Artificial Sequence
  Description of Artificial Sequence: Synthetic oligonucleotide
ggatctcgacgctctccctggaggacctggaccgtgtc

SEQ ID NO: 22 - DNA, 37 bp, Artificial Sequence
  Description of Artificial Sequence: Synthetic oligonucleotide
ggatctcgacgctctccctcctcaggggacgactccg

SEQ ID NO: 23 - DNA, 53 bp, Artificial Sequence
  Description of Artificial Sequence: Synthetic oligonucleotide
acactctttccctacacgacgctcttccgatctcttgcaggatgggccggtga

SEQ ID NO: 24 - DNA, 61 bp, Artificial Sequence
  Description of Artificial Sequence: Synthetic oligonucleotide
acactctttccctacacgacgctcttccgatctcatctctttgtcggtggtattaactcca

SEQ ID NO: 25 - DNA, 52 bp, Artificial Sequence
  Description of Artificial Sequence: Synthetic oligonucleotide
acactctttccctacacgacgctcttccgatctacaggggcgaggtcatcac

SEQ ID NO: 26 - DNA, 53 bp, Artificial Sequence
  Description of Artificial Sequence: Synthetic oligonucleotide
acactctttccctacacgacgctcttccgatcttggatgcactggagtcagca

SEQ ID NO: 27 - DNA, 54 bp, Artificial Sequence
  Description of Artificial Sequence: Synthetic oligonucleotide
acactctttccctacacgacgctcttccgatctctctcccaggggtttgcctaa

SEQ ID NO: 28 - DNA, 61 bp, Artificial Sequence
  Description of Artificial Sequence: Synthetic oligonucleotide
acactctttccctacacgacgctcttccgatctgagatcaggtctgacaagtctttctctg

SEQ ID NO: 29 - DNA, 61 bp, Artificial Sequence; identical to SEQ ID NO: 28
  Description of Artificial Sequence: Synthetic oligonucleotide

SEQ ID NO: 30 - DNA, 54 bp, Artificial Sequence
  Description of Artificial Sequence: Synthetic oligonucleotide
acactctttccctacacgacgctcttccgatctgagggtcaccgcatggacaag

SEQ ID NO: 31 - DNA, 53 bp, Artificial Sequence
  Description of Artificial Sequence: Synthetic oligonucleotide
acactctttccctacacgacgctcttccgatctcatcgtggccttgacctcca

SEQ ID NO: 32 - DNA, 53 bp, Artificial Sequence
  Description of Artificial Sequence: Synthetic oligonucleotide
acactctttccctacacgacgctcttccgatctgccaatggcggactcaaacg

SEQ ID NO: 33 - DNA, 51 bp, Artificial Sequence
  Description of Artificial Sequence: Synthetic oligonucleotide
acactctttccctacacgacgctcttccgatctctgcaggatgggccggtg

SEQ ID NO: 34 - DNA, 55 bp, Artificial Sequence
  Description of Artificial Sequence: Synthetic oligonucleotide
acactctttccctacacgacgctcttccgatctagctccttgtcggtggtgttag

SEQ ID NO: 35 - DNA, 51 bp, Artificial Sequence
  Description of Artificial Sequence: Synthetic oligonucleotide
acactctttccctacacgacgctcttccgatctcgtcagcctccaccagct

SEQ ID NO: 36 - DNA, 55 bp, Artificial Sequence
  Description of Artificial Sequence: Synthetic oligonucleotide
acactctttccctacacgacgctcttccgatctccagtggtgtgttggagctcat

SEQ ID NO: 37 - DNA, 51 bp, Artificial Sequence
  Description of Artificial Sequence: Synthetic oligonucleotide
acactctttccctacacgacgctcttccgatctcccaaggggcttgcccag

SEQ ID NO: 38 - DNA, 54 bp, Artificial Sequence
  Description of Artificial Sequence: Synthetic oligonucleotide
acactctttccctacacgacgctcttccgatctccgacaggtccttgtcagtgg

SEQ ID NO: 39 - DNA, 51 bp, Artificial Sequence
  Description of Artificial Sequence: Synthetic oligonucleotide
acactctttccctacacgacgctcttccgatctcccgccaactgcacacac

SEQ ID NO: 40 - DNA, 54 bp, Artificial Sequence
  Description of Artificial Sequence: Synthetic oligonucleotide
acactctttccctacacgacgctcttccgatctgtgtccttaccgtgacgtcca

SEQ ID NO: 41 - DNA, 52 bp, Artificial Sequence
  Description of Artificial Sequence: Synthetic oligonucleotide
acactctttccctacacgacgctcttccgatctcgactccgtgtttgcccac

SEQ ID NO: 42 - DNA, 64 bp, Artificial Sequence
  Description of Artificial Sequence: Synthetic oligonucleotide
aatgatacggcgaccaccgagatctacactagatcgcacactctttccctacacgacgctcttc

SEQ ID NO: 43 - DNA, 64 bp, Artificial Sequence
  Description of Artificial Sequence: Synthetic oligonucleotide
aatgatacggcgaccaccgagatctacacctctctatacactctttccctacacgacgctcttc

SEQ ID NO: 44 - DNA, 64 bp, Artificial Sequence
  Description of Artificial Sequence: Synthetic oligonucleotide
aatgatacggcgaccaccgagatctacactatcctctacactctttccctacacgacgctcttc

SEQ ID NO: 45 - DNA, 64 bp, Artificial Sequence
  Description of Artificial Sequence: Synthetic oligonucleotide
aatgatacggcgaccaccgagatctacacagagtagaacactctttccctacacgacgctcttc

SEQ ID NO: 46 - DNA, 64 bp, Artificial Sequence
  Description of Artificial Sequence: Synthetic oligonucleotide
aatgatacggcgaccaccgagatctacacgtaaggagacactctttccctacacgacgctcttc

SEQ ID NO: 47 - DNA, 64 bp, Artificial Sequence
  Description of Artificial Sequence: Synthetic oligonucleotide
aatgatacggcgaccaccgagatctacacactgcataacactctttccctacacgacgctcttc

SEQ ID NO: 48 - DNA, 64 bp, Artificial Sequence
  Description of Artificial Sequence: Synthetic oligonucleotide
aatgatacggcgaccaccgagatctacacaaggagtaacactctttccctacacgacgctcttc

SEQ ID NO: 49 - DNA, 64 bp, Artificial Sequence
  Description of Artificial Sequence: Synthetic oligonucleotide
aatgatacggcgaccaccgagatctacacctaagcctacactctttccctacacgacgctcttc

SEQ ID NO: 50 - DNA, 66 bp, Artificial Sequence
  Description of Artificial Sequence: Synthetic oligonucleotide
caagcagaagacggcatacgagattcgccttagtgactggagttcagacgtgtgctcttccgatct

SEQ ID NO: 51 - DNA, 66 bp, Artificial Sequence
  Description of Artificial Sequence: Synthetic oligonucleotide
caagcagaagacggcatacgagatctagtacggtgactggagttcagacgtgtgctcttccgatct

SEQ ID NO: 52 - DNA, 66 bp, Artificial Sequence
  Description of Artificial Sequence: Synthetic oligonucleotide
caagcagaagacggcatacgagatttctgcctgtgactggagttcagacgtgtgctcttccgatct

SEQ ID NO: 53 - DNA, 66 bp, Artificial Sequence
  Description of Artificial Sequence: Synthetic oligonucleotide
caagcagaagacggcatacgagatgctcaggagtgactggagttcagacgtgtgctcttccgatct

SEQ ID NO: 54 - DNA, 66 bp, Artificial Sequence
  Description of Artificial Sequence: Synthetic oligonucleotide
caagcagaagacggcatacgagataggagtccgtgactggagttcagacgtgtgctcttccgatct

SEQ ID NO: 55 - DNA, 66 bp, Artificial Sequence
  Description of Artificial Sequence: Synthetic oligonucleotide
caagcagaagacggcatacgagatcatgcctagtgactggagttcagacgtgtgctcttccgatct

SEQ ID NO: 56 - DNA, 66 bp, Artificial Sequence
  Description of Artificial Sequence: Synthetic oligonucleotide
caagcagaagacggcatacgagatgtagagaggtgactggagttcagacgtgtgctcttccgatct

SEQ ID NO: 57 - DNA, 66 bp, Artificial Sequence
  Description of Artificial Sequence: Synthetic oligonucleotide
caagcagaagacggcatacgagatcctctctggtgactggagttcagacgtgtgctcttccgatct

SEQ ID NO: 58 - DNA, 66 bp, Artificial Sequence
  Description of Artificial Sequence: Synthetic oligonucleotide
caagcagaagacggcatacgagatagcgtagcgtgactggagttcagacgtgtgctcttccgatct

SEQ ID NO: 59 - DNA, 66 bp, Artificial Sequence
  Description of Artificial Sequence: Synthetic oligonucleotide
caagcagaagacggcatacgagatcagcctcggtgactggagttcagacgtgtgctcttccgatct

SEQ ID NO: 60 - DNA, 66 bp, Artificial Sequence
  Description of Artificial Sequence: Synthetic oligonucleotide
caagcagaagacggcatacgagattgcctcttgtgactggagttcagacgtgtgctcttccgatct

SEQ ID NO: 61 - DNA, 66 bp, Artificial Sequence
  Description of Artificial Sequence: Synthetic oligonucleotide
caagcagaagacggcatacgagattcctctacgtgactggagttcagacgtgtgctcttccgatct

SEQ ID NO: 62-65 - skipped (4 entries)

(19) United States
(12) Patent Application Publication
(10) Pub. No.: US 2015/0337364 A1
(43) Pub. Date: Nov. 26, 2015

(54) Isothermal Methods and Related Compositions for Preparing Nucleic Acids

(71) Applicant: ARCHERDX, INC., Boulder, CO (US)

(72) Inventors: Joshua STAHL, Boulder, CO (US); Jason MYERS, Golden, CO (US)

(21) Appl. No.: 14/605,030

(22) Filed: Jan. 26, 2015

Related U.S. Application Data
(60) Provisional application No. 61/931,974, filed on Jan. 27, 2014.

(51) Int. Cl. C12Q 1/68

(52) U.S. Cl. CPC C12Q 1/6844; C12Q 1/6806; C12Q 1/6869

(57) ABSTRACT

According to some aspects of the invention preparative methods and related compositions are provided for nucleic acid sequencing.

### BACKGROUND OF INVENTION

Recent advances in next generation sequencing technologies have led to a rapid increase in sequencing and preparatory methods in both research and clinical settings. The high-throughput capability and high coverage depth make next generation sequencing an attractive and promising direction for molecular diagnostics. In lieu of whole genome sequencing, specific subsets of genes can be interrogated and multiple samples can be pooled (e.g., multiplexed) into a single sequencing run (e.g., flow cell lane), thus reducing the overall cost of analysis. Current methods are still rate limiting and improved methods are desirable.

### SUMMARY OF INVENTION

Aspects of the invention relate to a recognition that existing methods for preparing nucleic acids for sequencing are both labor intensive and often require large quantities of starting material. It has been further recognized that existing methods involve steps (e.g., ligation steps, end repair and polyadenylation-tailing) that are both lengthy and inefficient, rendering the methods ineffective for obtaining rapid and accurate sequencing results, which is desirable in a molecular diagnostic context. In some embodiments, methods herein provide rapid amplification of template nucleic acids under isothermal conditions to produce samples that can be directly used with standard next generation nucleic acid sequencing systems, including, for example, high throughput flow cell based sequencing systems. In some embodiments, aspects of the invention relate to methods for preparing nucleic acids that involve exponentially amplifying nucleic acids under isothermal conditions for sequencing. Thus, in some embodiments, methods provided herein are advantageous because they employ isothermal reaction conditions, circumventing the need for specialized thermal cycling machinery. In some embodiments, methods provided herein are advantageous because they can be employed using RNA and/or DNA as a starting material. Thus, in some embodiments, methods can be employed using nucleic acids extracted from a variety of different types of samples (e.g., blood and other tissue samples), including samples that are obtained for pathological analysis, thereby enabling parallel diagnostic tests to be performed on common tissue samples.

In some embodiments, it has been recognized that conventional preparative methods often not only rely on thermal cycling (e.g., as with PCR), but also on invariant, known sequences in order to design amplification primers that flank a target region. In some embodiments, this limits the types of genetic events captured using existing methods, making it a challenge to detect nucleic acid variants resulting from hypermutation, gene rearrangements or fusions with unknown genetic partners. Accordingly, in some embodiments, methods provided herein are useful for preparing nucleic acids for sequencing for purposes of detecting of a wide-range of genetic variants, rearrangements or polymorphisms. For example, methods are provided, in some embodiments, that are advantageous for amplifying nucleic acid templates that contain known target sequences fused to unknown sequences for purposes of identifying the unknown sequences. Thus, in some embodiments, methods provided herein are useful for preparing nucleic acid fusions resulting from genetic rearrangements. In some embodiments, the fusions are mRNA fusions that are encoded in genes that have undergone a chromosomal rearrangement. In some embodiments, the fusions are chromosomal segments comprising two loci that have been fused together as a result of a chromosomal rearrangement. Thus, in some embodiments, preparative methods provided herein are useful for amplifying pools of nucleic acids for purposes of sequencing the nucleic acids to detect genomic rearrangements or fusions. In some embodiments, methods provided herein may be used to detect genomic rearrangements or fusions using sequencing as a complementary diagnostic test for a standard pathological assay, e.g., a fluorescent in situ hybridization assay. In some embodiments, preparative methods provided herein are useful for de novo gene assembly, e.g., shot gun sequencing. In such embodiments, oligonucleotides with hybridization sequences may be used to amplify nucleic acids that comprise junctions between genome assembly fragments (e.g., between contigs). Accordingly, in some embodiments, preparative methods may be used to confirm the correctness of a genome assembly prediction by amplifying nucleic acids that comprise junctions between genome assembly fragments, determining the actual sequence on either side of the fragments and confirming whether the actual sequences fit with genome assembly predictions.

Aspects of the invention relate to methods of preparing a nucleic acid for analysis. In some embodiments, the methods involve (a) producing a synthetic RNA from a nucleic acid template; (b) exponentially amplifying the synthetic RNA in an isothermal reaction; and (c) generating a eDNA from the exponentially amplified synthetic RNA, wherein the cDNA comprises at least one non-target sequence. Further aspects of the invention relate to methods of determining a sequence of a nucleic acid template. In some embodiments, the methods involve (a) producing a synthetic RNA from a nucleic acid template; (b) exponentially amplifying the synthetic RNA in an isothermal reaction; (c) generating a cDNA from the exponentially amplified synthetic RNA; and (d) sequencing the cDNA. In certain embodiments, the exponential amplification of step (b) is repeated. In some embodiments, the amplified synthetic RNA is purified after each consecutive round of step (b), and the purified synthetic RNA is used as starting material for the subsequent round(s) of step (b). In certain embodiments, at least two of the isothermal reactions of repeated step (h) comprise template-dependent extensions that are primed by oligonucleotides having hybridization sequences that are complementary with nested sequences of the template synthetic RNA or first DNA strand.

In some embodiments, the isothermal reaction comprises two or more cycles of template-dependent extension and RNA polymerase transcription. In certain embodiments, at least one template-dependent extension in each cycle is a reverse transcription. In some embodiments, the isothermal reaction is performed at a temperature in range of 35° to 45° C. In certain embodiments, the isothermal reaction is performed for a duration of 45 to 90 minutes. In some embodiments, the isothermal reaction comprises a template-dependent extension that synthesizes a first DNA strand that is complementary to the synthetic RNA, resulting in formation of a RNA-DNA hybrid between the first DNA strand and the synthetic RNA. In certain embodiments, the isothermal reaction further comprises degradation of the synthetic RNA portion of the RNA-DNA hybrid. In some embodiments, the degradation is enzymatically mediated degradation. In certain embodiments, the degradation is mediated by RNAse H. In some embodiments, the isothermal reaction further comprises a template-dependent extension that synthesizes a second DNA strand that is complementary to the first DNA, resulting in formation of a double-stranded DNA comprising the first and second DNA strands. In some embodiments, the isothermal reaction further comprises an RNA polymerase mediated transcription reaction that transcribes synthetic RNAs from the double-stranded DNA.

In some embodiments, at least two of the isothermal reactions of repealed step (b) comprise template-dependent extensions that are primed by oligonucleotides having hybridization sequences that are complementary with the template synthetic RNA or first DNA strand and additional non-complementary sequences. In certain embodiments, the additional non-complementary sequences comprises one or more of a barcode sequence, an index sequence, or an adapter sequence.

In some embodiments, the methods further comprise producing the nucleic acid template by performing at least one extension reaction using a oligonucleotide that comprises a target-specific hybridization sequence; and performing at least one extension reaction using a plurality of different oligonucleotides that share a common sequence that is 5′ to different hybridization sequences.

In certain embodiments, the nucleic acid template comprises a target region and an adjacent region. In some embodiments, the target-specific hybridization sequence is complementary with the target region and wherein at least one of the different hybridization sequences is complementary with the adjacent region. In certain embodiments, the target region comprises a sequence of a first gene and the adjacent region comprises a sequence of a second gene. In some embodiments, the first gene is RET, ROS1 or ALK.

In certain embodiments, the nucleic acid template is a double-stranded DNA comprising a promoter, wherein the synthetic RNA is enzymatically produced through an RNA polymerase that specifically binds to the promoter and transcribes DNA downstream of the promoter. In some embodiments, the RNA polymerase is a T3, T7, or SP6 polymerase. In certain embodiments, the synthetic RNA is transcribed from an intermediate double-stranded DNA produced from the nucleic acid template, wherein the nucleic acid template is an isolated RNA. In some embodiments, the isolated RNA is a messenger RNA (mRNA), microRNA, ribosomal RNA, transfer RNA, or non-coding RNA. In certain embodiments, the mRNA is fusion mRNA encoded from a chromosomal segment that comprises a genetic rearrangement. In some embodiments, the nucleic acid template is a chromosomal segment that comprises a portion of a genetic rearrangement. In certain embodiments, the genetic rearrangement is an inversion, deletion, or translocation. In some embodiments, the cDNA contains a don-template sequence that serves as a hybridization site for a sequencing primer that primes the sequencing reaction. In certain embodiments, the cDNA is sequenced in a multiplex reaction that includes different nucleic acids originating from different sources. In some embodiments, the different sources are different subjects from which the nucleic acid templates were obtained. In certain embodiments, the different sources are different tissues from which the nucleic acid templates were obtained.

Further aspects of the invention relate to methods of sequencing a nucleic acid. In some embodiments, the methods involve producing a synthetic RNA from a nucleic acid template that comprises a target region and an adjacent region; producing a double-stranded nucleic acid that comprises a first strand synthesized by a template-dependent extension using the synthetic RNA as a template and a second strand synthesized by a template-dependent extension using the first strand as a template, wherein the double-stranded nucleic acid is representative of the target region and the adjacent region of the nucleic acid template; and performing a sequencing reaction using the double-stranded nucleic acid to determine a nucleotide sequence of the target region and the adjacent region. In some embodiments, the methods further comprise amplifying the synthetic RNA and producing the double-stranded nucleic acid using the amplified synthetic RNA as a template. In some embodiments, the synthetic RNA is amplified by an isothermal amplification. In certain embodiments, the synthetic RNA is exponentially amplified by the isothermal amplification. In some embodiments, the synthetic RNA is amplified by polymerase chain reaction.

In certain embodiments, the methods further comprise amplifying the double-stranded nucleic acid and sequencing the amplified double-stranded nucleic acid. In some embodiments, each strand of the double-stranded nucleic acid is produced such that is contains a non-template sequence that serves as a hybridization site for a sequencing primer that primes the sequencing reaction. In certain embodiments, the double-stranded nucleic acid is sequenced in a multiplex reaction that includes different nucleic acids originating from different sources. In some embodiments, the different nucleic acids comprise source identifying barcode sequences.

Further aspects of the invention relate to kits comprising components that are useful in methods disclosed herein. In some embodiments, the kits comprise a container housing a lyophilized composition that comprises at least one oligonucleotide comprising a hybridization sequence and RNA polymerase promoter sequence; a reverse transcriptase; a DNA polymerase; and an RNA polymerase. In some embodiments, the composition further comprises an RNAse H. In certain embodiments, the reverse transcriptase is selected from the group consisting of AMV reverse transcriptase, RSV reverse transcriptase, HIV-1 reverse transcriptase, and HIV-2 reverse transcriptase. In some embodiments, the DNA polymerase is selected from the group consisting of: Taq polymerase, Pheonix Taq polymerase, Phusion polymerase, T4 polymerase, T7 polymerase, Klenow fragment, Klenow exo-, phi29 polymerase, VeraSeq ULtra polymerase, and EnzScript. In certain embodiments, the RNA polymerase is selected from the group consisting of: T3 polymerase, T7 polymerase, and SP6 polymerase. In some embodiments, the at least one oligonucleotide further comprises at least one of a barcode sequence, an index sequence and an adapter sequence. In certain embodiments, the container is a chamber of a multichamber cartridge.

### BRIEF DESCRIPTION OF DRAWINGS

FIGS. 1A-G depict a non-limiting example of a work flow for isothermal amplification of target nucleic acid sequences that are flanked by a 3′ unknown fusion partner beginning with RNA as a template.

FIGS. 2A-E show a non-limiting example of a work flow for the isothermal amplification of target nucleic acid sequences that are flanked by a 5′ unknown sequence beginning with RNA as a template.

FIGS. 3A-D depict a non-limiting example of a work flow for the isothermal amplification of target nucleic acid sequences using DNA as a template.

FIGS. 4A-E depict a non-limiting example of a work flow for the isothermal amplification of target nucleic acid sequences that are flanked by either 5′ or 3′ unknown sequences beginning with RNA as a template.

### DETAILED DESCRIPTION OF INVENTION

Methods herein enable rapid preparation of template nucleic acids to produce samples that can be directly used with standard nucleic acid sequencing systems, including, for example, high throughput flow cell based sequencing systems. In some embodiments, preparative methods are provided that involve exponentially amplifying nucleic acids under isothermal conditions for sequencing. Thus, in some embodiments, methods provided herein are advantageous because they employ isothermal reaction conditions, circumventing the need for specialized thermal cycling machinery. In some embodiments, methods are provided for preparing nucleic acids for sequencing that involve alternating template-dependent extension and RNA transcription reactions performed under isothermal conditions to exponentially amplify template nucleic acids. Moreover, in some embodiments, preparative methods disclosed herein can produce amplified nucleic acids for sequencing within approximately 2 to 5 hours, thus enabling relatively rapid molecular diagnostic testing by sequencing. Moreover, methods provided herein enable parallel testing (e.g., by sequencing and image-based analysis) to be performed on common tissue samples. For example, in some embodiments, methods for preparing nucleic acids disclosed herein are suitable for use with nucleic acid extracted from biological samples (e.g., formalin fixed tissue sections, blood and other tissues) that are obtained for pathological analysis.

It will be understood that methods provided herein have a number of applications, including, without limitation, preparing nucleic acids for partial or complete nucleotide sequencing. In some embodiments, methods provided herein are advantageous because they can be employed using a range of different nucleic acids as starting materials, including RNA or DNA. In some embodiments, methods disclosed herein involve preparing nucleic acids that are representative of chromosomal segments that exist in a genome, including mammalian genomes and more particularly human genomes. Nucleic acids prepared using methods disclosed herein may include subsets of a genome (such as exons or an exome), a transcriptome or subsets thereof, or other DNA or RNA obtained from cells.

In some embodiments, methods disclosed herein involve preparing nucleic acids for purposes of determining the presence or absence of a nucleic acid in a sample by sequencing. Such methods can be useful in, for example, diagnostic and forensic applications. In some embodiments, methods disclosed herein involve preparing nucleic acids for purposes of determining whether a nucleic acid comprises a mutation or variation in sequence, such as, for example an allelic variation, including a single nucleotide polymorphism, a genetic rearrangement, a copy number variation and so on. In some embodiments, methods disclosed herein involve preparing nucleic acids for purposes of determining the presence of a genetically modified organisms or genetically engineered nucleic acids in a sample.

In some embodiments, methods disclosed herein are useful for preparing nucleic acids from any appropriate sample (e.g., a food sample, environmental sample, biological sample e.g., blood sample, etc.) for purposes of detecting and/or sequencing nucleic acids present in the sample. In some embodiments, the nucleic acids may be prepared to facilitate sequence based detection of a pathogen, infectious agent, or organism in the sample. The term “food sample” refers to any liquid, semisolid, solid and dry material that is edible, including e.g., meat and meat products, milk and milk-based products, eggs and egg-based products, bakery products, confectionaries, vegetables, fruit and beverages including drinking water, etc. Environmental samples include samples of surface water, ground water, ocean water, soil samples and air samples, etc. The term “biological sample” includes any cell, tissue, biological fluid, organ or portion thereof. A biological sample may be obtained or derived, for example, from cells or tissue cultures in vitro. Alternatively, a biological sample may be obtained or derived from an organism. Examples, include blood, sputum, urine, biopsies (e.g., tumor biopsies) and other samples that are normally tested in clinical laboratories. The term biological sample also includes samples that have been processed for analysis, e.g., a fixed tissue section.

In some embodiments, methods disclosed herein involve preparing nucleic acids for purposes of determining by sequencing or other detection method whether a known nucleic acid has undergone mutation that results in disease (e.g., cancer). In some embodiments, methods disclosed herein involve preparing nucleic acids for purposes of determining the presence or absence of a particular condition in a subject by sequencing the nucleic acids. The condition may be cancer, a non-cancerous condition, such as a neurodegenerative condition, or an infection, for example. In some embodiments, methods disclosed herein involve preparing nucleic acids for purposes of evaluating genetic differences that exist between two samples, such as, for example, normal tissue and diseased tissue. In some embodiments, methods disclosed herein involve preparing nucleic acids for purposes of determining by sequencing or other method the carrier status of an individual. In some embodiments, methods disclosed herein involve preparing nucleic acids from prenatal samples for purposes of prenatal genetic testing. Methods disclosed herein can be useful for preparing samples for determining by sequencing an underlying cause of antibiotic resistance in microorganisms or immune or antiviral resistance in the context of viruses.

Moreover, in certain embodiments of the methods, multiple reactions may be carried out in parallel for purposes of processing or evaluating multiple nucleic acids and/or samples from multiple sources (e.g., multiple patient samples). For example, 10-25, 15-50, 25-75, 50-100, 75-200, 100-500, 200-500, 200-1000, 500-1500, 1000-2500, 2500-5000 or more nucleic acids (e.g., different genetic loci, e.g., different fusion breakpoints or polymorphisms) may be evaluated for each sample. It should be appreciate that multiple reactions may be performed in a single reaction chamber or separate reaction chambers. Moreover, samples from multiple different sources may be processed in parallel. For example, 1-25, 25-50, 50-100, 100-500, 500-1000, 1000-2500, 2500-5000 or more or intermediate numbers of sources may be processed in parallel.

It should be appreciated that methods disclosed herein may be automated and/or may involve the use of robotics for carrying out reactions or transferring materials between reactions. For example, in an automated implementation, nucleic acids prepared using a preparative method disclosed herein may be transferred to a sequencing platform for sequencing using robotics or other automated components. Furthermore, sequencing data obtained from a detector or a sensor of a sequencing system may be input to a computer, mobile device, and/or displayed on a screen so that a user can monitor progress of the sequencing reactions remotely or access and analyze information obtained from the sequencing reactions.

In some embodiments, nucleic acids prepared by methods disclosed herein are analyzed through nucleic acid sequencing. In some embodiments, the nucleic acid sequencing is a next generation sequencing method. In some embodiments the next generation sequencing method is a sequencing by synthesis method as applicable to the Illumina next generation sequencers in which adapter sequences flanking the amplified DNA to be sequenced contain appropriate sequences for this method. In some embodiments, the sequencing method uses an ion semiconductor as applicable to the Ion Torrent sequencing platform wherein adapter sequences flanking the amplified DNA to be sequenced contain the appropriate sequences for this method, Additional sequencing methods for nucleic acid analysis include but are not limited to chain-termination sequencing (also referred to as Sanger sequencing), sequencing by ligation (also referred to as SOLiD sequencing), 454 pyrosequencing, and single-molecule real-time sequencing (also referred to as Pacific Biosciences sequencing).

In some embodiments, sequencing by synthesis (e.g., using an Illumina system) involves use of adapter sequences (P5, P7) that are joined to either end of a nucleic acid to be analyzed and are complementary to the P5 and P7 oligonucleotides that are immobilized within a flow cell. In some embodiments, the process involves clonal amplification of the immobilized DNA molecules followed by addition of fluorescently-labeled nucleotides that are incorporated into the complementary DNA strand as it is synthesized one cycle at a time. In addition to the P5 and P7 adapter sequences, the amplified DNA may also contain sequences for hybridization with one or more sequencing oligonucleotide (e.g., sequences referred to as Rd1 or Rd2).

In some embodiments, ion semiconductor sequencing methods (e.g., using an Ion Torrent system) involve distinct adapter sequence (A, P1) that are joined to either end of a nucleic acid to be analyzed and allow attachment of the nucleic acid molecules to a sphere particles. In some embodiments, the sphere-particle-conjugated nucleic acid is amplified by emulsion PCR (emPCR) and loaded into chip wells for sequencing. The ion semiconductor sequencer is based on detection of protons released during polymerization of a DNA strand that is complementary to the particle-conjugated template DNA. Each released hydrogen ion is detected by a hypersensitive ion sensor.

In some embodiments, methods provided herein involve joining additional sequences to a target nucleic acid through amplification of the target sequences. In some embodiments, oligonucleotides contain hybridization sequences for hybridizing with a template nucleic acid and additional sequences. In some embodiments, the additional sequences comprise one or more of the following non-limiting examples including identifier sequences (e.g., barcodes), sequencing primer hybridization sequences (e.g., Rd1), adapter sequences, and others. In some embodiments adapter sequences are sequences involve in an analysis with a next generation sequencing technology. In some embodiments, adapter sequences are P5 (SEQ ID NO: 62) and/or P7 (SEQ ID NO: 63) sequences for Illumina-based sequencing technology. In some embodiments, adapter sequences are P1 (SEQ ID NO: 64) and A (SEQ ID NO: 65) compatible with Ion Torrent sequencing technology.

In some embodiments, methods are provided for preparing nucleic acids encompassing genetic rearrangement events that have occurred between a genetic region of interest and an unknown fusion partner. Thus, more generally, methods are provided herein for preparing and evaluating nucleic acids that have a target region next to an adjacent region (e.g., the sequence of which adjacent region is to be determined). In some cases, the target region is a region of a known gene (e.g., an oncogene) that is a hotspot for genetic rearrangements that give rise to fusion proteins that cause disease. Thus, in some embodiments, methods described herein may be used to identify both the location of the fusion event as well as the sequence of an unknown fusion partner. In some embodiments, methods provided herein can be used to amplify genetic rearrangements that have occurred 3′ of a known target sequence. In some embodiments, methods can be used to amplify genetic rearrangements that have occurred 5′ of a known target locus. In other embodiments, the methods can be used to identify inversions, deletion or translocation events. In some embodiments, a target nucleic acid is a messenger RNA. In some embodiments, the target nucleic acid is a chromosomal DNA segment. Methods provided herein to can be used to prepare nucleic acids encompassing these rearrangements at the DNA level by isolating genomic DNA and amplifying loci containing the breakpoints associated with these fusions. In other embodiments, methods provided herein can be used to prepare nucleic acids encompassing these rearrangements at the RNA level by isolating cellular RNA and amplifying fusion mRNAs encoded in loci containing these rearrangements. In some embodiments, the methods may be used to evaluate RET, ROS1, FGFR3 and ALK fusions associated with cancer.

The following table provides a further non-limiting list of examples of genetic rearrangements that may be interrogated using methods provided herein.

TABLE 1
Oncogenes Resulting from Chromosomal Rearrangements

| Oncogene | Function/Activation | Cancer* |
|---|---|---|
| AF4/HRX | Fusion affects the HRX transcription | Acute leukemias |
| | factor/methyltransferase. HRX is also called | |
| | MLL, ALL1 and HTRX1 | |
| ALK/NPM | Translocation creates fusion protein with | Large cell lymphomas |
| | nucleophosmin(NPM) | |
| AML1/MTG8 | New fusion protein created by translocation | Acute leukemias |
| BCR/ABL | New protein created by fusion of BCR and | Chronic myelogenous and acute |
| | ABL triggers unregulated cell growth | lymphotic leukemia |
| DEK/CAN | Fusion protein | Acute myeloid leukemia |
| E2A/PBX1 | Fusion protein | Acute pre B-cell leukemia |
| ENL/HRX | Fusion protein created by a translocation | Acute leukemias |
| | t(11; 19). | |
| ERG/TLS | Fusion protein created by t(16:21) | Myeloid leukemia |
| | translocation. The ERG protein is a | |
| | transcription factor. | |
| EWS/FLI-1 | Fusion protein created by t(11:22) | Ewing Sarcoma |
| | translocation. | |
| FGFR3- | Fusion protein | Bladder cancer |
| TACC3 | | |
| KIF5B-RET | Fusion protein | NSCLC |
| LYT-10/C | Fusion protein formed by the (10; 14)(q24; q32) | |
| ALPHA1 | translocation of LYT-10 next to the C ALPHA1 | |
| | immunoglobulin locus. | |
| MTG8/AML1 | Fusion of transcription repressor to factor to a | Acute leukemias |
| | transcription factor. AML1 is also known as | |
| | RUNX1. | |
| MYH11/CBFB | New protein created by fusion of transcription | Acute myeloid leukemia |
| | factors via an inversion in chromosome 16. | |
| PBX1/E2A | Fusion protein formed via t(1:19) | Acute pre B-cell leukemia |
| | translocation. Transcription factor. | |
| PIST-ROS | Resulting from an intra-chromosomal | Glioblastoma multiform |
| | homozygous deletion of 240 kilobases on 6q21 | |
| RAR/PML | Fusion protein caused by t(15:17) | Acute premyelocytic leukemia |
| | translocation. Retinoic acid receptor. | |
| REL/NRG | Fusion protein formed by deletion in | B-cell lymphoma |
| | chromosome 2. Transcription factor. | |
| SET/CAN | Fusion protein formed by rearrangement of | Acute myeloid leukemia |
| | chromosome 9. Protein localization | |

*The cancer types listed in this column are those that are predominantly associated with each oncogene but this is not a complete list.

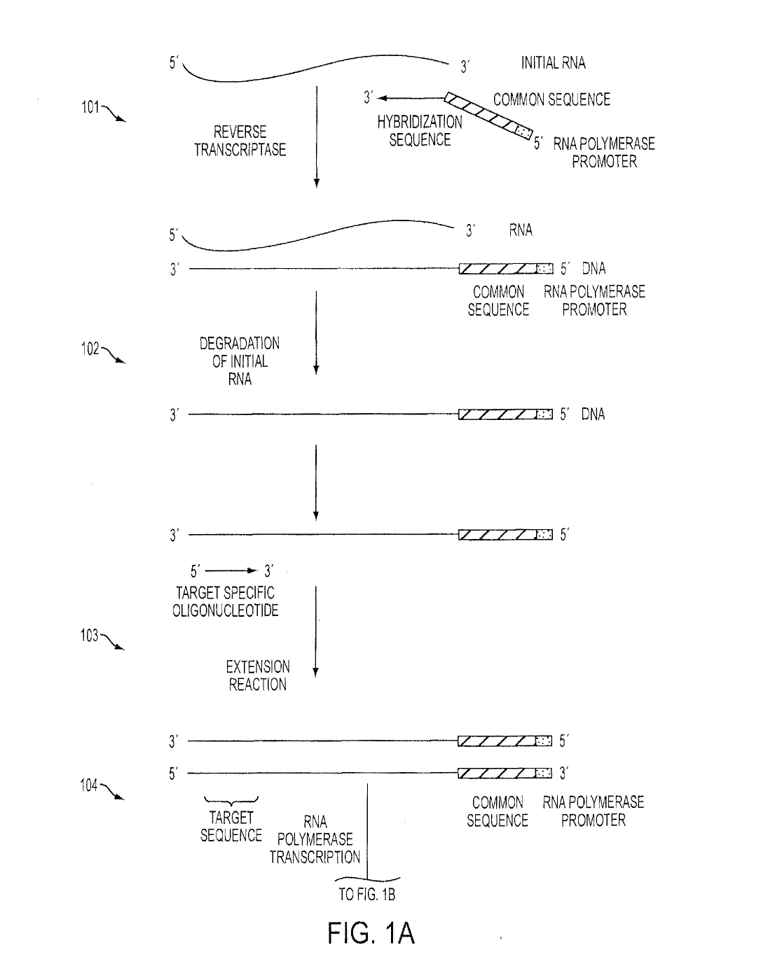

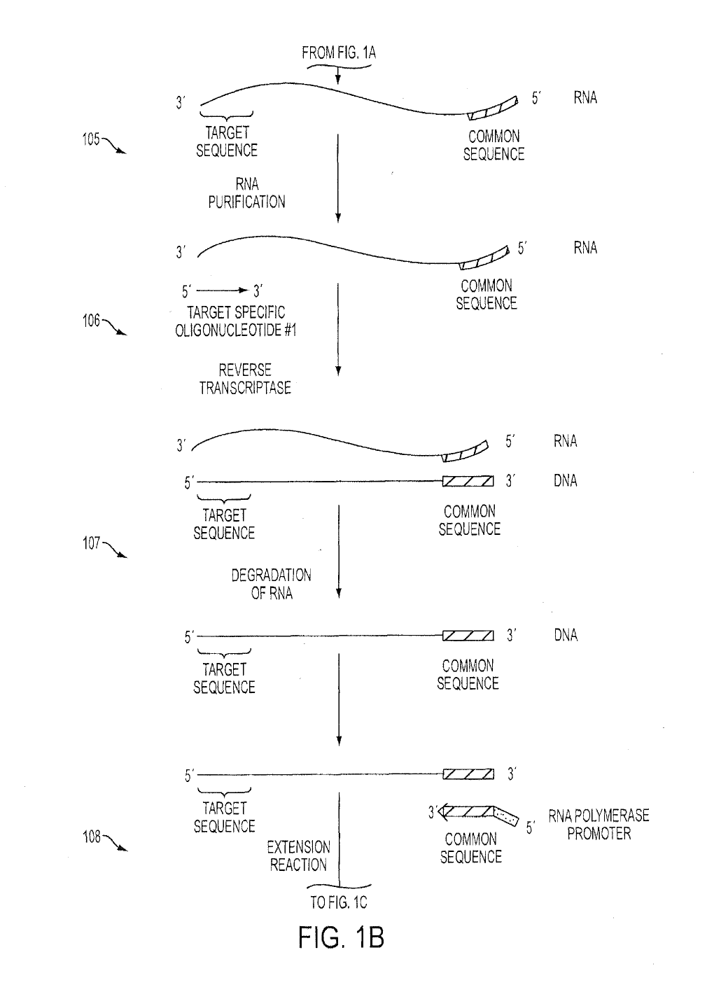

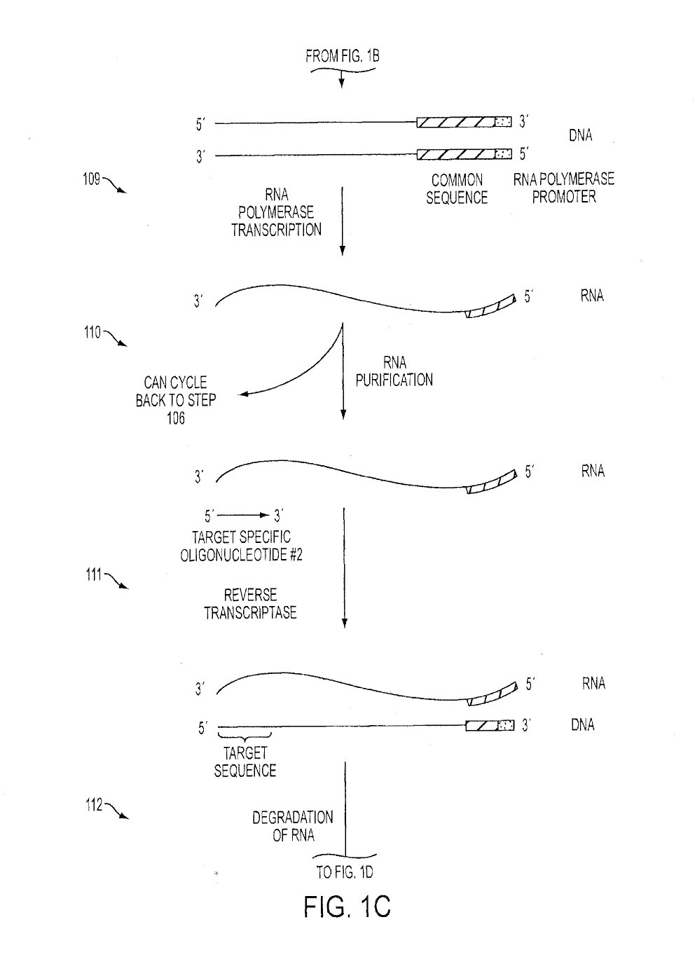

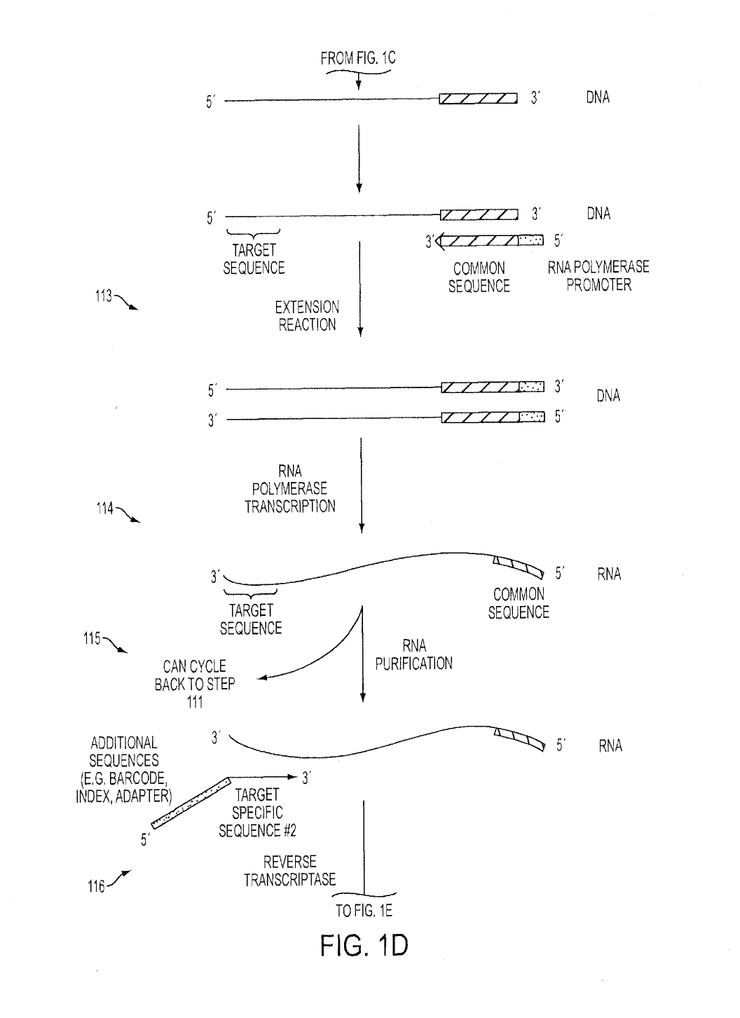

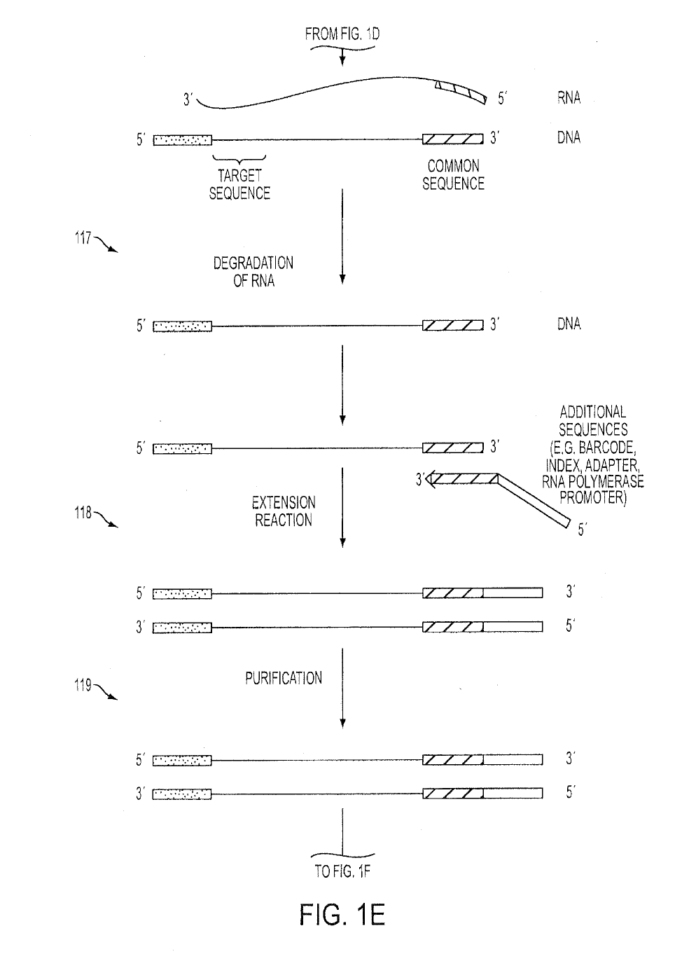

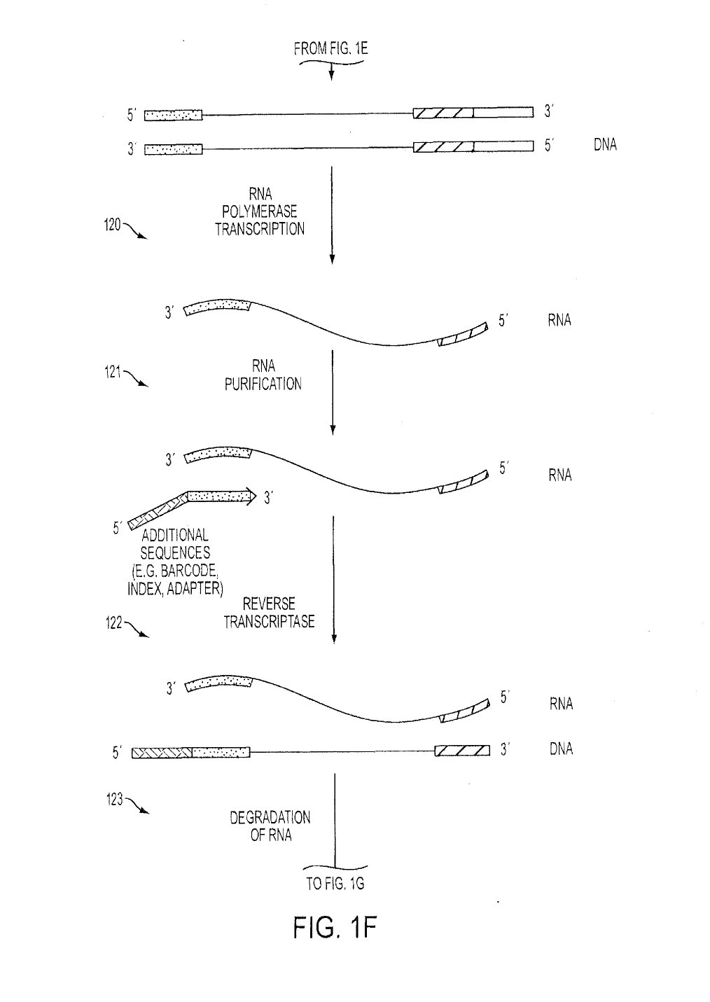

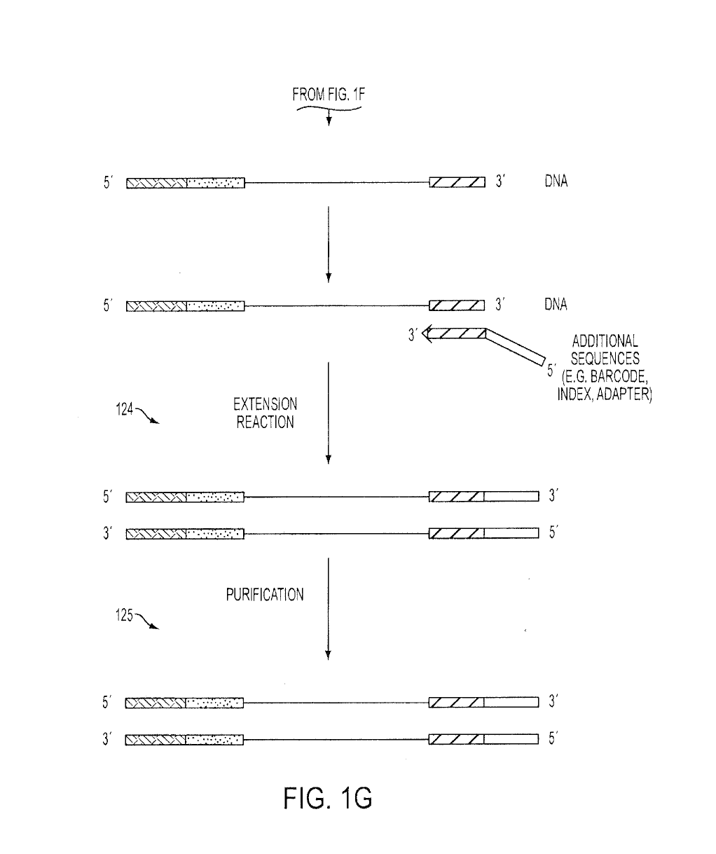

In some embodiments, methods are provided for preparing nucleic acids that have a target region 5′ to an adjacent region (e.g., an adjacent region of unknown sequence content). For example, FIG. 1 illustrates an exemplary process for preparing nucleic acids having a target region 5′ to an adjacent region. At step 101, an initial RNA is obtained or provided as a template molecule. The RNA template is exposed to a plurality of oligonucleotides that share a common sequence that is 5′ to different hybridization sequences. In some embodiments, the common sequence shared by the plurality of oligonucleotides also contains an RNA polymerase promoter sequence. In some embodiments, at least one of the hybridization sequences hybridizes to a region of the RNA template and functions to prime a first reverse transcriptase reaction to produce a DNA molecule that is complementary to the initial RNA. At step 102, the initial RNA template is enzymatically degraded (e.g., by RNase H) from the hybrid RNA-DNA molecule. While it is appreciated that RNase H is used in the provided example, many enzymes with RNase activity may be used, as described herein.

In step 103, the remaining DNA molecule is contacted by one or more target-specific oligonucleotides such that a target-specific oligonucleotide hybridizes to a region of the DNA and is extended to synthesize a complementary DNA strand. In some embodiments this reaction is performed by Phoenix DNA polymerase. In some embodiments this reaction is performed by a dual function reverse transcriptase that also has DNA polymerase activity (e.g., AMV reverse transcriptase enzyme). However, it should be appreciated that other appropriate polymerase enzymes may be used, as described herein. In step 104, RNA polymerase transcribes, using the RNA polymerase promoter contained within the common sequence, an RNA molecule complementary to the DNA template. In some embodiments, steps 101-104 can be repeated through multiple cycles of each of which begins with the complementary RNA molecule resulting from step 104 serving as the template at step 101. The transcribed RNA may be subsequently purified in step 105.

In step 106, the purified RNA containing the 5′ common sequence is then contacted by one or more target-specific oligonucleotides. Target-specific oligonucleotide #1 hybridizes with the complementary RNA at a target sequence and primes a template-dependent reverse transcriptase reaction producing a complementary DNA strand. In step 107, the RNA template is enzymatically degraded (e.g., through the RNase activity) from the complementary hybrid RNA-DNA molecule of RNaseH. In step 108, the remaining DNA molecule is contacted by an oligonucleotide containing a sequence encoding an RNA polymerase promoter 5′ to a sequence complementary to the common sequence present on the 3′ end of the DNA molecule. The oligonucleotide is extended through the activity of a DNA polymerase in reaction to produce a complementary DNA strand. In step 109, RNA polymerase utilizes the RNA polymerase promoter to transcribe a complementary RNA molecule. Steps 106-109 are repeated through multiple cycles each of which begins with the complementary RNA molecule resulting from step 109 serving as the template at step 106, thus amplifying the RNA at step 109.

It should be appreciated that, in some embodiments, the number of cycles through 106-109 is influenced at least in part by the duration of the isothermal reaction. Furthermore, in some embodiments, as the process is cycled through step 109, the DNA template produced accumulates such that the last cycle results in an exponentially amplified pool of RNA molecules relative to the starting material. RNA molecules from reaction 109 may also be purified as in step 110 in preparation for subsequent steps. In some embodiments, steps 101-109 are performed consecutively in a single reaction tube. In some embodiments, all of the components involve in steps 101-109 are present at the outset and throughout the reaction. In some embodiments, steps 101-109 are performed as isothermal reactions.

Optionally, a second cycle of amplification may be performed in which the RNA molecules purified in step 110 are contacted by one or more target-specific oligonucleotide. In step 111, the target-specific oligonucleotide hybridizes with the complementary RNA at a target sequence and primes a template-dependent reverse transcriptase reaction producing a complementary DNA strand. In step 112, the RNA template is enzymatically degraded from the complementary DNA strand (e.g., by RNaseH). In step 113, the remaining DNA molecule is contacted by an oligonucleotide containing a sequence encoding an RNA polymerase promoter 5′ to a sequence complementary to the common sequence present on the 3′ end of the DNA molecule. The oligonucleotide is extended through DNA polymerase activity to produce a complementary DNA strand. In step 114, RNA polymerase utilizes the RNA polymerase promoter to transcribe a complementary RNA molecule. Steps 111-114 are repeated through multiple cycles each of which begins with the complementary RNA molecule resulting from step 114 serving as the template at step 111. RNA molecules from step 114 may also be purified as in step 115 in preparation for subsequent steps. In some embodiments, further cycles of amplification may be performed if desired.

In step 116, the purified RNA is contacted by one or more target-specific oligonucleotides #2 comprising a target-specific sequence and additional sequences 5′ to the target-specific sequence that may include a common sequence, barcode, index, or adapter sequences. The target-specific oligonucleotide hybridizes with the complementary RNA at a target sequence and primes the reverse transcriptase reaction producing a complementary DNA strand also containing the 5′ additional sequences provided by target-specific oligonucleotide #2. In some embodiments, target-specific oligonucleotide #1 and target-specific oligonucleotide #2 contain distinct sequences. In some embodiments the sequence of target-specific oligonucleotide #2 is present within the template DNA molecule 3′/downstream of the sequence of target-specific oligonucleotide #1 such that the reactions are nested.

In step 117, the RNA template strand is enzymatically degraded (e.g., by RNase H). In step 118, the remaining DNA molecule is contacted by an oligonucleotide containing a sequence complementary to the common sequence present on the 3′ end of the DNA molecule as well as an additional sequence that may contain any one or more sequences including barcode, index, and adapter sequences. The oligonucleotide hybridizes and is extended to produce a complementary DNA strand. The resulting DNA molecule is double stranded and contains the target sequence and its adjacent region flanked by additional sequences that contain adapter sequences for the appropriate sequencing platform. The product is purified in reaction 119 and ready for analysis. Optionally, the additional sequences provided on the oligonucleotide in step 118 may contain a RNA polymerase promoter 5′ to the complementary common sequence. In this case, following the extension reaction of step 118, in step 120 RNA polymerase utilizes the RNA polymerase promoter to transcribe a complementary RNA molecule. Steps 116-118 are repeated through multiple cycles each of which begins with the complementary RNA molecule resulting from step 118 serving as the template at step 116 RNA molecules from step 120 may also be purified as in step 121 in preparation for subsequent steps.

Further amplification cycles may be performed to add additional sequences to one or both ends of the nucleic acid. In step 122, an oligonucleotide with a sequence complementary to the 3′ end of the RNA molecule hybridizes with the RNA and primes a template-dependent reverse transcriptase reaction producing a complementary DNA strand. In some embodiments, the oligonucleotide of step 122 contains additional sequences. In some embodiments the additional sequence comprise barcode, index, and/or adapter sequences. In step 123, the RNA template is enzymatically degraded from the complementary DNA strand (e.g., by RNase H). In step 124, the remaining DNA molecule is contacted by an oligonucleotide containing a sequence complementary to the common sequence present on the 3′ end of the DNA molecule as well as an additional sequence that may contain any one or more sequences including barcode, index, and adapter sequences. In some embodiments, the oligonucleotide hybridizes and is extended to produce a complementary DNA strand that is purified in step 125. In some embodiments, the resulting DNA molecule is double stranded and contains the target sequence and its adjacent region flanked by additional sequences that contain adapter sequences for the appropriate sequencing platform.

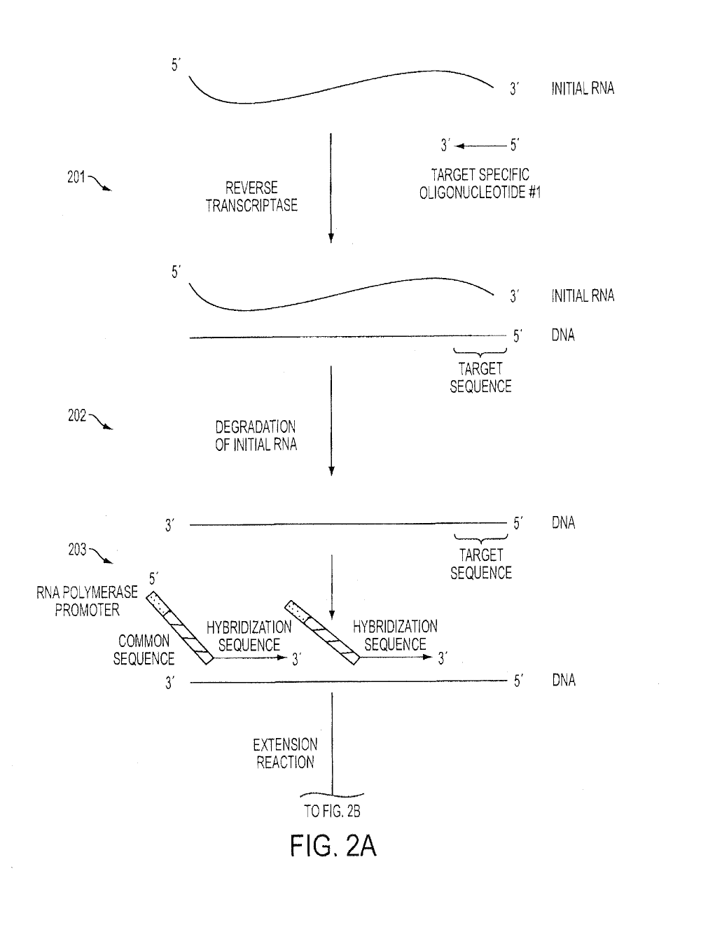

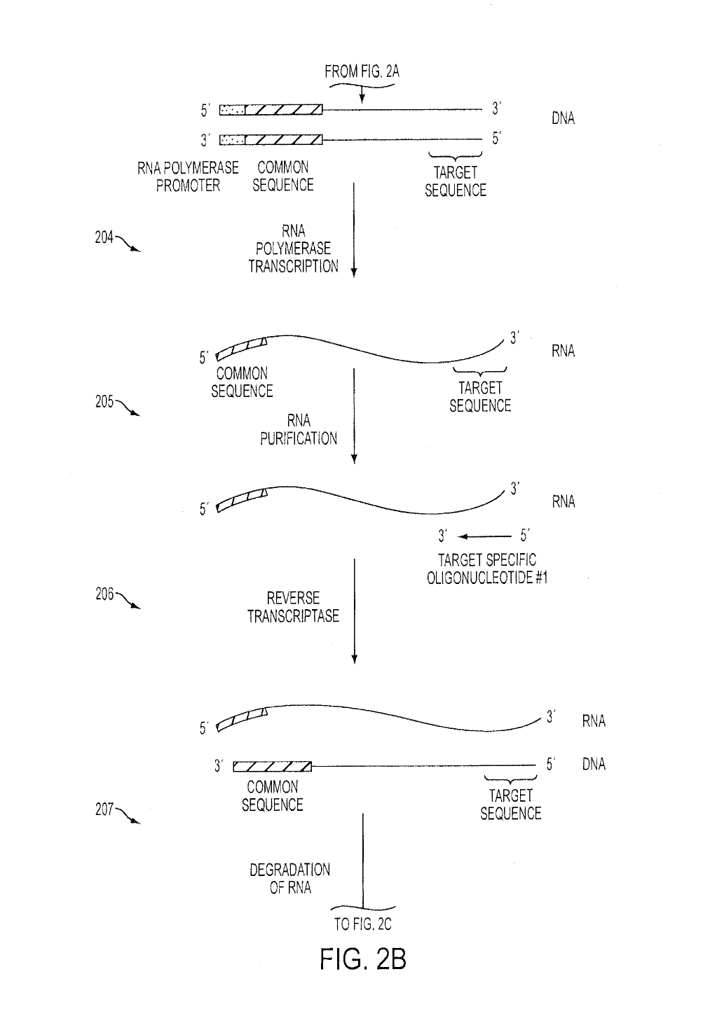

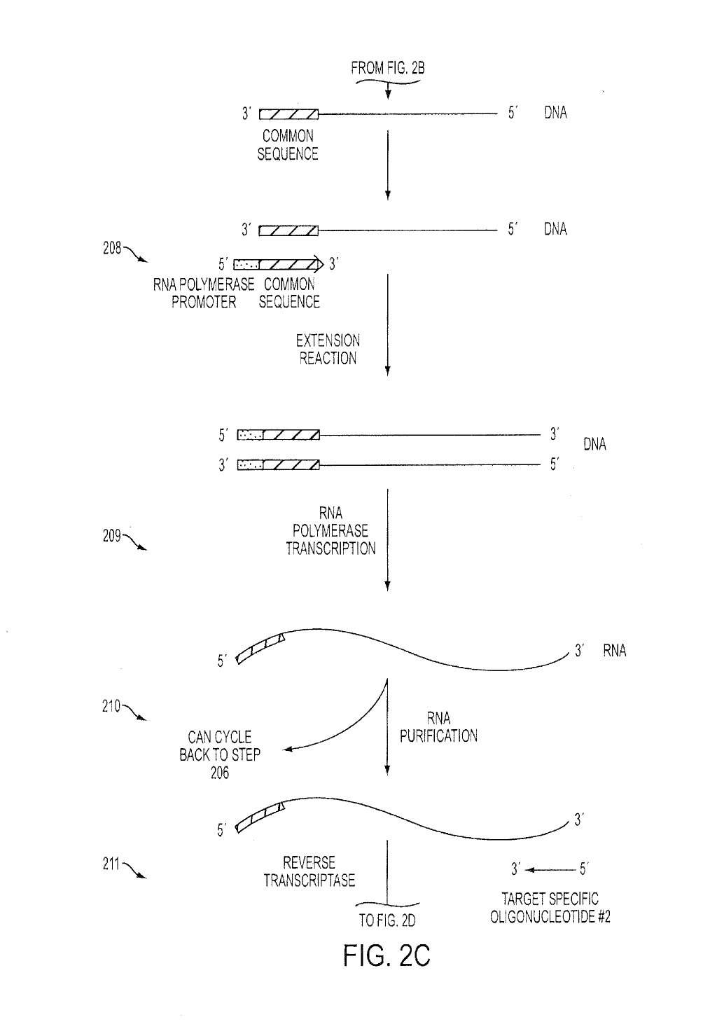

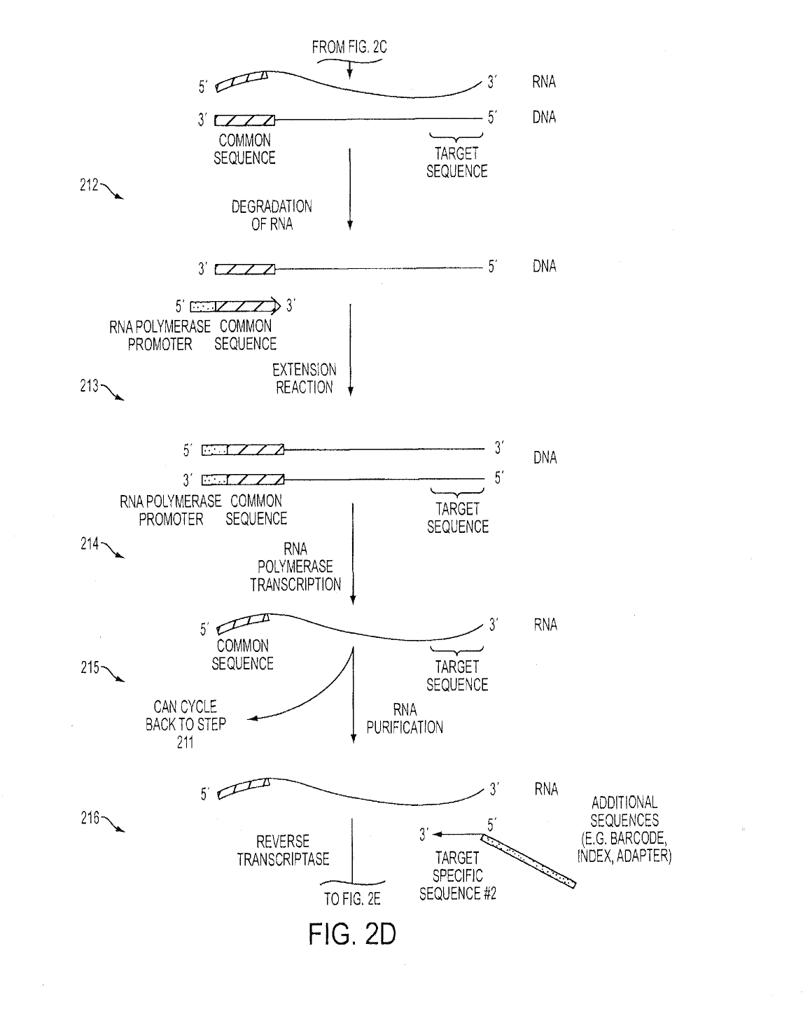

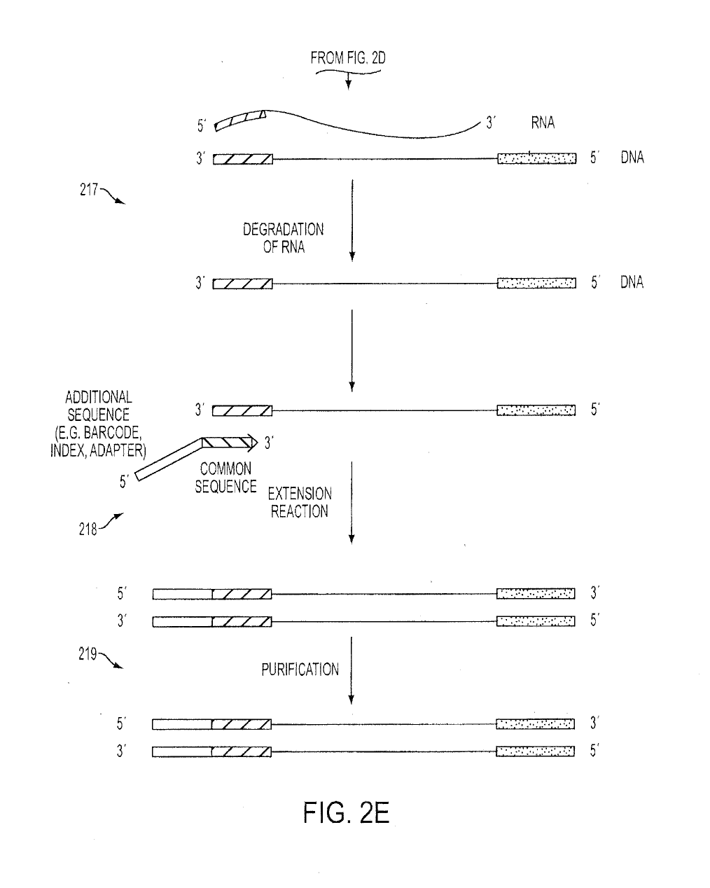

In some embodiments, methods are provided for preparing nucleic acids that have a target region 3′ to an adjacent region (e.g., an adjacent region of unknown sequence content). For example, FIG. 2 illustrates an exemplary process for preparing nucleic acids having a target region that is 3′ to an adjacent region. An initial RNA is obtained or provided as a template molecule. In step 201, the RNA template is exposed to one or more target-specific oligonucleotides, referred to as target-specific oligonucleotide #1, that is complementary to a target region of the initial RNA. The target-specific oligonucleotide #1 hybridizes and primes a first reverse transcriptase reaction to produce a DNA molecule that is complementary to the initial RNA. In step 202, the initial RNA template is enzymatically degraded (e.g., by RNaseH) from the complementary hybrid RNA-DNA molecule. In step 203, the remaining DNA molecule is contacted by a plurality of oligonucleotides that share a common sequence that is 5′ to different hybridization sequences (e.g., random or pseudorandom sequences, sets of different predefined sequences, etc.). In some embodiments, the common sequence shared by the plurality of oligonucleotides also contains an RNA polymerase promoter sequence. In some embodiments, at least one of the hybridization sequences hybridizes to a region of the DNA and is extended to synthesize a second complementary DNA strand. In step 204, RNA polymerase transcribes, using the RNA polymerase promoter sequence, an RNA molecule complementary to the DNA template. The transcribed RNA is subsequently purified in step 205.

In step 206, the purified RNA containing the 5′ common sequence is then contacted by one or more target-specific oligonucleotide #1. The target-specific oligonucleotide hybridizes with the complementary RNA at a target sequence and primes a template-dependent reverse transcriptase reaction producing a complementary DNA strand. In reaction 207, the RNA template is enzymatically degraded (e.g., RNaseH activity) from the complementary hybrid RNA-DNA molecule. In step 208, the remaining DNA molecule is contacted by an oligonucleotide containing a sequence encoding an RNA polymerase promoter 5′ to a sequence complementary to the common sequence present on the 3′ end of the DNA molecule. The oligonucleotide is extended to produce a complementary DNA strand. In step 209, RNA polymerase utilizes the RNA polymerase promoter to transcribe a complementary RNA molecule. Steps 206-209 are repeated through multiple cycles each of which begins with the complementary RNA molecule resulting from step 209 serving as the template at step 206. RNA molecules from reaction 209 may also be purified as in step 210 in preparation for subsequent steps. In some embodiments, steps 201-209 are performed consecutively in a single reaction tube. In some embodiments, steps 201-209 are performed as isothermal reactions.

Optionally, a second cycle of amplification may be performed in which the RNA molecule purified in step 210 are contacted by one or more target-specific oligonucleotide #2. In some embodiments, the use of one or more target-specific oligonucleotides adds specificity and further enriches for nucleic acids comprising target sequences. In step 211, the target-specific oligonucleotide #2 hybridizes with the complementary RNA at a target sequence and primes a template-dependent reverse transcriptase reaction producing a complementary DNA strand. In step 212, the RNA template is enzymatically degraded (e.g., RNaseH). In step 213 the remaining DNA molecule is contacted by an oligonucleotide containing a sequence encoding an RNA polymerase promoter 5′ of a sequence complementary to the common sequence present on the 3′ end of the DNA molecule. The oligonucleotide is extended through DNA polymerase activity to produce a complementary DNA strand. In some embodiments, DNA activity is provided by a dual function enzyme, e.g., AMV reverse transcriptase. In step 214, RNA polymerase transcribes a complementary RNA molecule. Steps 211-214 are repeated through multiple cycles each of which begins with the complementary RNA molecule resulting from step 214 serving as the template at step 211. RNA molecules produced in step 214 may also be purified in step 215. In some embodiments, further cycles of amplification may be performed if desired.

In step 216, the purified RNA is contacted by one or more target-specific oligonucleotide #2 comprising a target-specific sequence and 5′ additional sequences that may include a common region, barcode, index, and/or adapter sequences. However, it should be appreciated that additional sequences can be incorporated using similar approaches at other points in the process. The target-specific oligonucleotide hybridizes with the complementary RNA at a target sequence and primers the reverse transcriptase reaction producing a complementary DNA strand also containing the 5′ additional sequences provided by target-specific oligonucleotide #2. In some embodiments, target-specific oligonucleotide #1 and target-specific oligonucleotide #2 contain distinct sequences. In some embodiments the sequence of target-specific oligonucleotide #2 is present within the template DNA molecule 3′/downstream of the sequence of target-specific oligonucleotide #1 such that the reactions are nested.

In step 217, the RNA template strand is enzymatically degraded (e.g., RNaseH). In reaction 218, the remaining DNA molecule is contacted by an oligonucleotide containing a sequence complementary to the common sequence present on the 3′ end of the DNA molecule as well as an additional sequence that may contain barcode, index, and/or adapter sequences. The oligonucleotide hybridizes and is extended to produce a complementary DNA strand. The resulting DNA molecule is double stranded and contains the target sequence and its adjacent sequence flanked by additional sequences that contain adapter sequences for the appropriate sequencing platform. This product is purified in step 219 and ready for analysis.

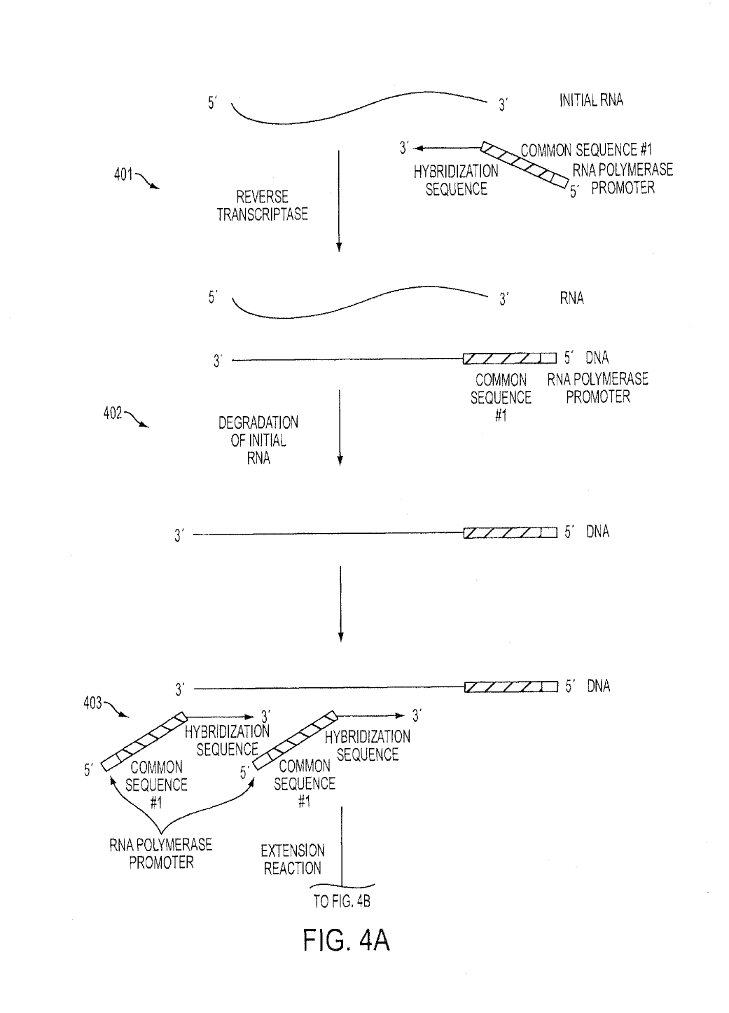

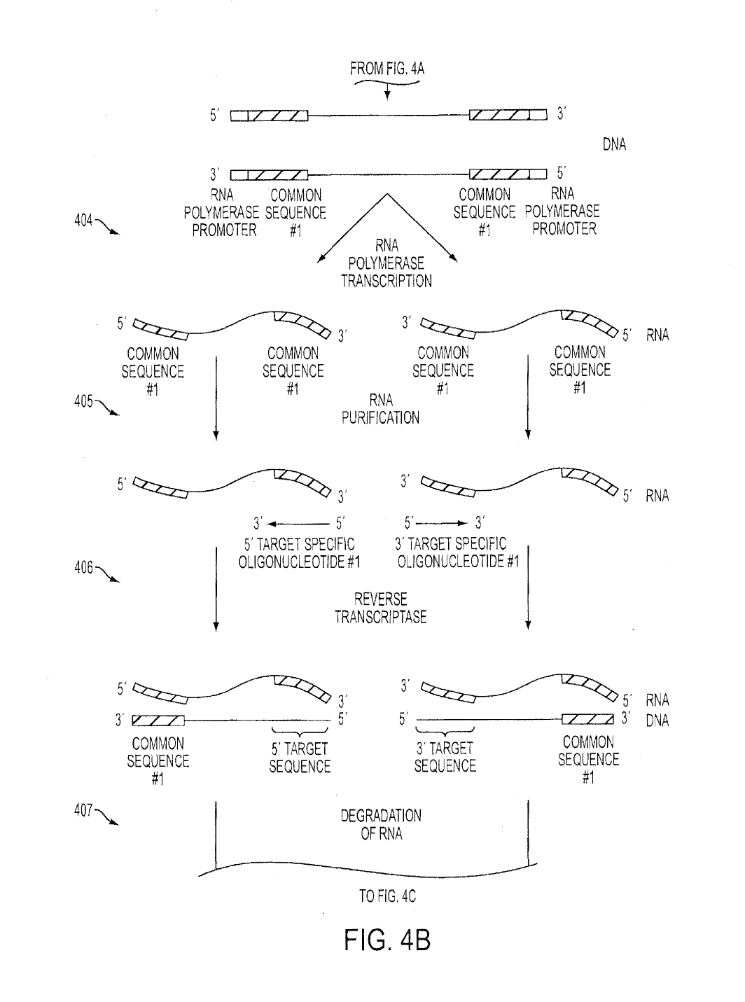

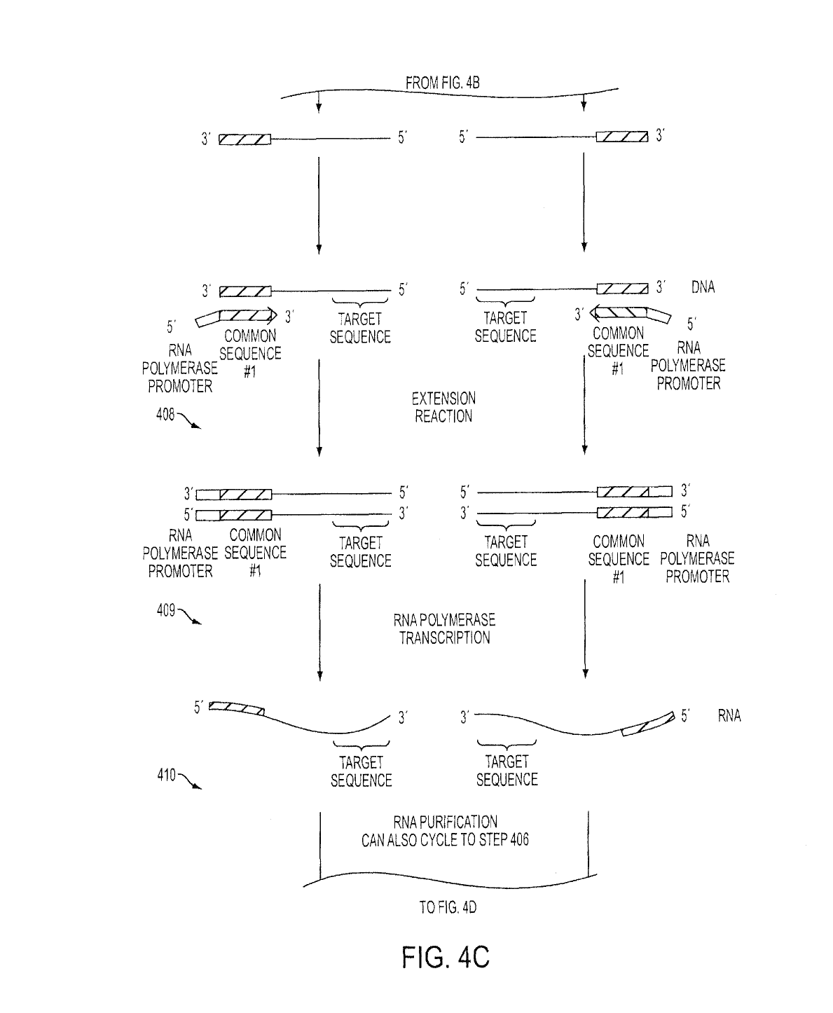

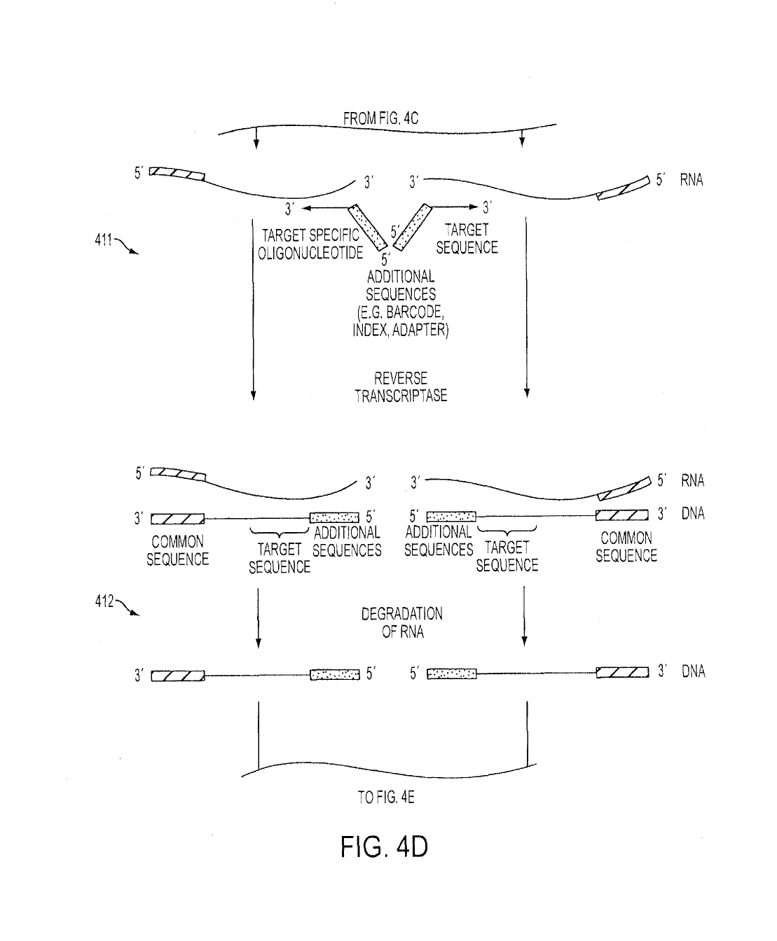

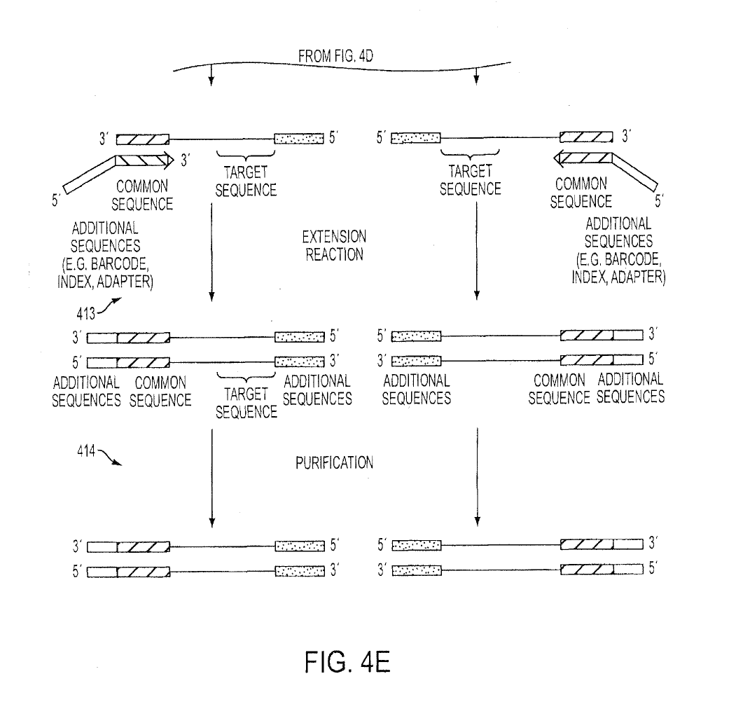

In some embodiments, methods depicted in FIGS. 1 and 2 may occur in parallel in the same reaction container (e.g., tube, cartridge well), e.g., in a manner similar to FIG. 4.

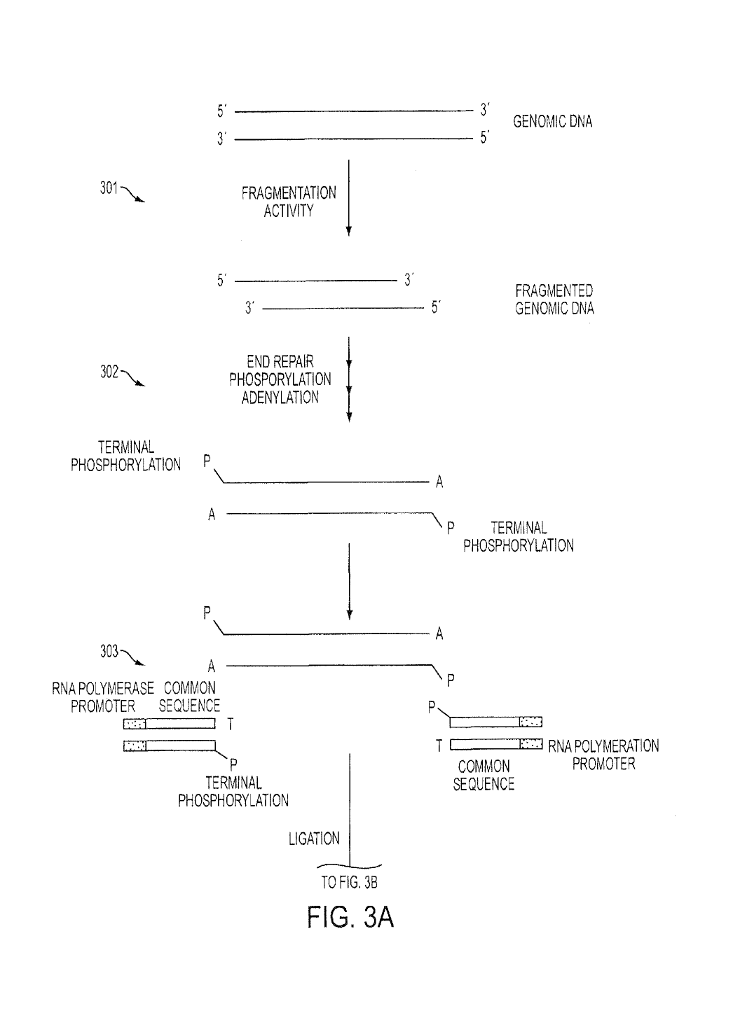

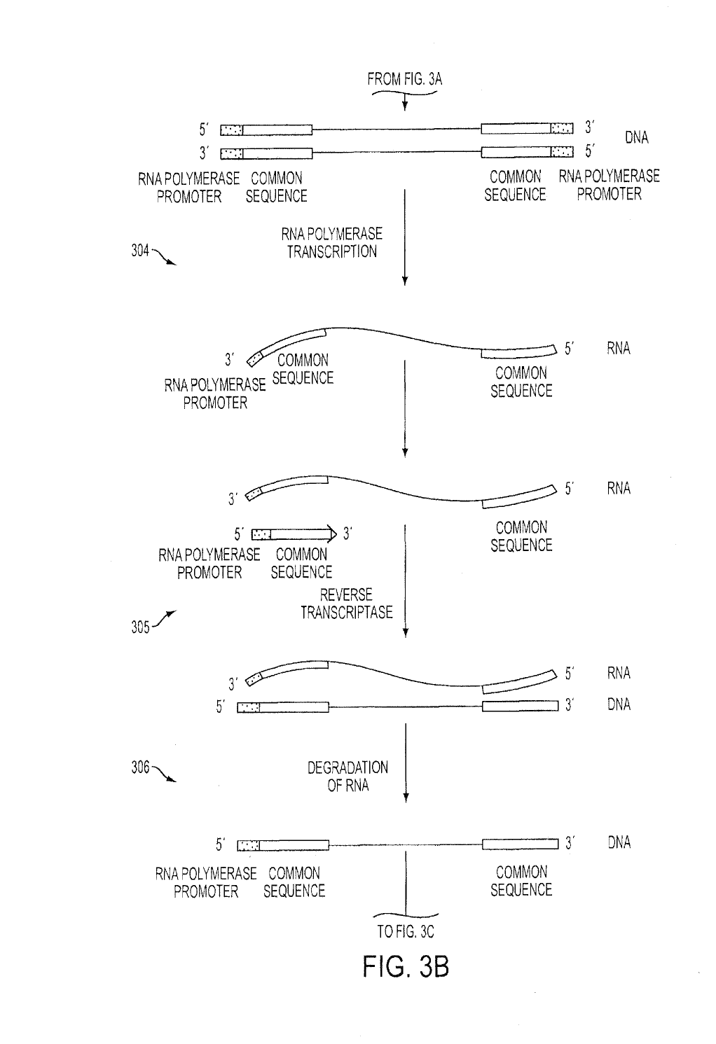

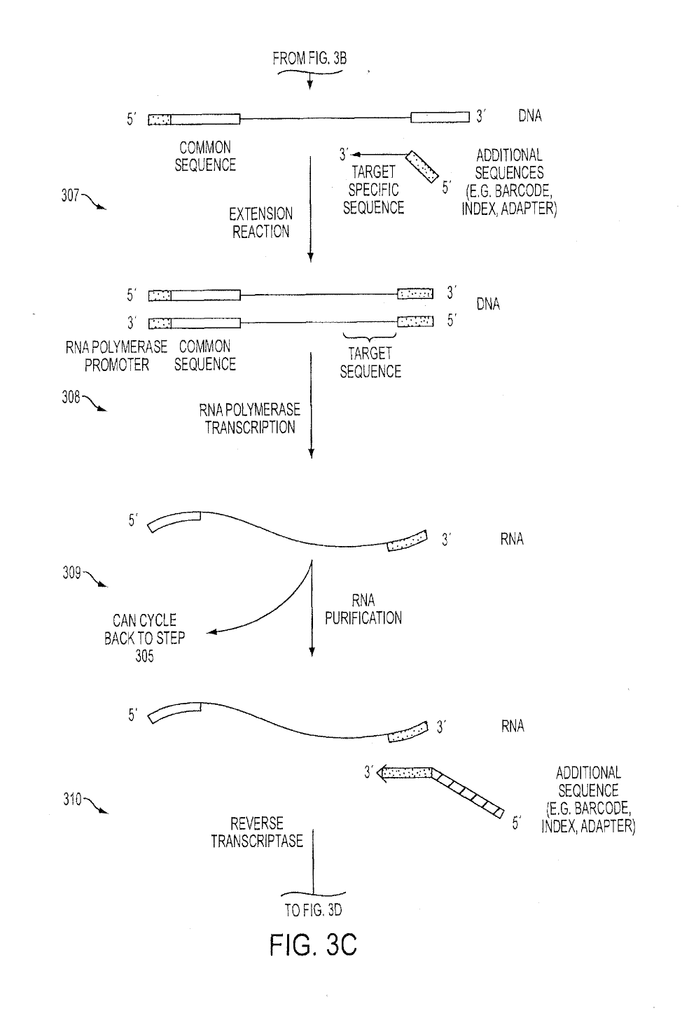

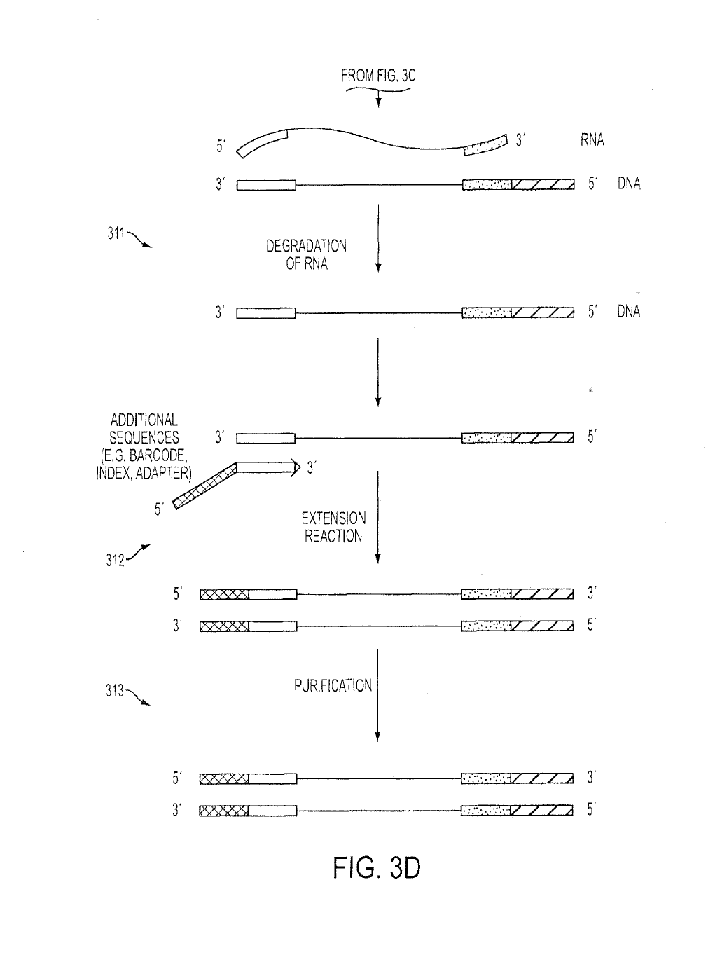

In some embodiments, methods are provided for preparing nucleic acids that comprise a target locus and an adjacent region using a DNA template. For example, FIG. 3 depicts an exemplary process for amplifying nucleic acid comprising a target locus and an adjacent region using a DNA template. An initial DNA is obtained or provided as a template molecule. In step 301, the DNA is disrupted into fragments (e.g., fragments of an appropriate length for sequencing, e.g., fragments ranging between 100-600, 100-1000, 100-1500 or more base pairs in length). In step 302, the ends of the fragmented DNA are repaired and a terminal phosphate group is added to each 5′ end. A single adenosine overhang is generated at each 3′ end through the activity of a terminal transferase, also in step 302. Using the terminal phosphate group and adenosine overhangs, double stranded adapters are ligated onto either end of the DNA fragment as in step 303. In some embodiments, the adapter molecules may contain a common sequence. In some embodiments, the adapter molecules also contain a RNA polymerase promoter sequence such that the ligation reaction results in a double stranded DNA molecule flanked on both ends by common and RNA polymerase promoter sequences.

In some embodiments, oligonucleotides (e.g., oligonucleotides that serve as adapter molecules) may contain one or more modifications to augment their stability and/or the stability of reaction products incorporating the oligonucleotides. Non-limiting examples of such modifications include based modifications and backbone modifications. In some embodiments, presence of phosphorothioatc bonds or other backbone modifications in a 3′ thymine overhang prevents 3′ exonuclease activity from blunt ending a oligonucleotide. In some embodiments, a bottom stand of an oligonucleotide adapter molecule can have an inverted deoxythymine that prevents PCR/AMP in subsequent steps from making products that are not gene specific

In step 304, RNA polymerases transcribe using one or both of the flanking RNA polymerase promoter sequences to produce complementary RNA molecules in one or both directions. In some embodiments, RNA is synthesized from both the positive and negative stands of the DNA molecule. In some embodiments, synthesis from both strands is advantageous because synthesized RNA may be extended using a target specific oligonucleotide to extend in either the 5′ or 3′ direction along a single DNA strand depending on the strand from which the RNA molecule is synthesized. Thus, in some embodiments, generating RNA from both positive and negative strands of the template molecule facilitates amplification and identification of unknown sequences adjacent in either direction to target sequences.

In step 305, the RNA molecule is contacted by an oligonucleotide comprising a sequence complementary to the common sequence on the RNA molecule. In some embodiments the oligonucleotide also contains a RNA polymerase promoter sequence. In step 305, the oligonucleotide hybridizes and primes the reverse transcriptase reaction producing a complementary DNA molecule. In step 306, the RNA template is enzymatically degraded (e.g., RNaseH) from the complementary hybrid RNA-DNA molecule. In step 307, the remaining DNA molecule is contacted by one or more target-specific oligonucleotides. In some embodiments the target-specific oligonucleotide contains additional sequences that may include barcode, index, and/or adapter sequences. The target-specific oligonucleotide hybridizes to a target region of the DNA and is extended producing a complementary DNA strand. In step 308, RNA polymerase transcribes a RNA molecule that is complementary to the DNA template. Steps 305-308 are repeated through multiple cycles each of which begins with the complementary RNA molecule resulting from step 308 serving as the template at step 305. RNA molecules from step 308 may also be purified in step 309 in preparation for subsequent steps. In some embodiments, steps 301-309 are performed consecutively in a single reaction tube. In some embodiments, steps 301-309 are performed as isothermal reactions.

In step 310, the purified RNA is contacted by a one or more target-specific oligonucleotide #2. In some embodiments, the target-specific oligonucleotide comprises additional sequences including barcode, index, and adapter sequences. The target-specific oligonucleotide #2 hybridizes and primes the reverse transcriptase reaction producing a complementary DNA molecule. In some embodiments, target-specific oligonucleotide #1 and target-specific oligonucleotide #2 contain the same sequence. In some embodiments, target-specific oligonucleotide #1 and target-specific oligonucleotide #2 contain distinct sequences. In some embodiments the sequence of target-specific oligonucleotide #2 is present within the template DNA molecule 3′/downstream of the sequence of target-specific oligonucleotide #1 such that the reactions are nested.

The DNA molecule resulting from step 310 may contain additional sequences provided on the target-specific oligonucleotide #2. In step 311, the RNA template is enzymatically degraded (e.g., RNase H). The remaining DNA molecule is contacted by an oligonucleotide containing a sequence complementary to the common sequence present on the 3′ end of the DNA molecule. In some embodiments the oligonucleotide contains any one or more of the additional sequences including barcode, index and adapter sequences. In step 312, the oligonucleotide hybridizes to the common sequence of the DNA molecule and is extended producing a complementary DNA strand. The DNA product of step 312 is double stranded and contains the target region and adjacent region flanked by additional sequences containing adapter sequences for the appropriate sequencing platform. This product is purified in step 313 and ready for analysis.

In some embodiments, methods are provided for preparing nucleic acids that have a target region that is flanked on its 5′ and/or 3′ end by an adjacent region (e.g., an adjacent region of unknown sequence content). For example, FIG. 4 illustrates an exemplary process for preparing nucleic acids having a target region with a 5′ and/or 3′ adjacent region. At step 401, an initial RNA is obtained or provided as a template molecule. The RNA template is exposed to a plurality of oligonucleotides that share a common sequence (common sequence #1) that is 5′ to different hybridization sequences. In some embodiments, the common sequence shared by the plurality of oligonucleotides also contains an RNA polymerase promoter sequence. In some embodiments, at least one of the hybridization sequences hybridizes to a region of the RNA template and functions to prime a first reverse transcriptase reaction to produce a DNA molecule that is complementary to the initial RNA. At step 402, the initial RNA template is enzymatically degraded (e.g., by RNase H) from the hybrid RNA-DNA molecule. While it is appreciated that RNase H is used in the provided example, many enzymes with RNase activity may be used, as described herein.

In step 403, the remaining DNA molecule is contacted by a plurality of oligonucleotides that share a common sequence that is 5′ to different hybridization sequences (e.g., random or pseudorandom sequences, sets of different predefined sequences, etc.). In some embodiments, the common sequence shared by the plurality of oligonucleotides also contains an RNA polymerase promoter sequence. In some embodiments, at least one of the hybridization sequences hybridizes to a region of the DNA and is extended to synthesize a second complementary DNA strand. In step 404, RNA polymerase transcribes, using the RNA polymerase promoter sequence, an RNA molecule complementary to the DNA template. In some embodiments, RNA polymerase promoters are present on both ends of the DNA template. In some embodiments both RNA polymerase promoters are utilized by RNA polymerase to generate both strands of complementary RNA. The transcribed RNA is subsequently purified in step 405.

In step 406, the purified RNA synthesized from one or both template strands is contacted by one or more target-specific oligonucleotide #1. The target-specific oligonucleotide hybridizes with the complementary RNA at a target sequence (e.g., at a common sequence) and primes a template-dependent reverse transcriptase reaction producing a complementary DNA strand. In reaction 407, the RNA template is enzymatically degraded (e.g., RNaseH activity) from the complementary hybrid RNA-DNA molecule. In step 408, the remaining DNA molecule is contacted by an oligonucleotide containing a sequence encoding an RNA polymerase promoter 5′ to a sequence complementary to the common sequence present on the 3′ end of the DNA molecule. The oligonucleotide is extended to produce a complementary DNA strand. In step 409, RNA polymerase utilizes the RNA polymerase promoter to transcribe a complementary RNA molecule. Steps 406-409 are repeated through multiple cycles each of which begins with the complementary RNA molecule resulting from step 409 serving as the template at step 406. RNA molecules from reaction 409 may also be purified as in step 410 in preparation for subsequent steps. In some embodiments, steps 401-409 are performed consecutively in a single reaction tube. In some embodiments, steps 401-409 are performed as isothermal reactions.

In step 411, the purified RNA by one or more target-specific oligonucleotide containing additional sequences 5′ to the target-specific sequence. In some embodiments, additional sequences include but are not limited to barcode, index, and/or adapter sequences. The target-specific oligonucleotide hybridizes with the complementary RNA at a target sequence and primes a template-dependent reverse transcriptase reaction producing a complementary DNA strand. In reaction 412, the RNA template is enzymatically degraded (e.g., RNase H activity) from the complementary hybrid RNA-DNA molecule. In step 413, the remaining DNA molecule is contacted by an oligonucleotide containing a sequence complementary to the common sequence present on the 3′ end of the DNA molecule. In some embodiments the oligonucleotide of step 413 contains additional sequences including but not limited to barcode, index, and/or adapter sequences. The oligonucleotide is extended to produce a complementary DNA strand that is purified in step 414, In some embodiments, the resulting DNA molecule is double stranded and contains the target sequence and its adjacent region flanked by additional sequences that contain adapter sequences for the appropriate sequencing platform (e.g., an Illumina platform, a Ion Torrent platform, etc.).

As used herein, the term “nucleic acid” refers to a polymeric molecule comprising a plurality of nucleotides covalently linked together by internucleotide linkages. In some embodiments, a nucleic acid is a ribonucleic acid (RNA) formed by a plurality of ribonucleotides covalently linked together by internucleotide linkages. In some embodiments, a nucleic acid is a deoxyribonucleic acid (DNA) formed by plurality of deoxyribonucleotides covalently linked together by internucleotide linkages. In some embodiments, a nucleic acid includes one or more nucleotide analogues (such a bridged nucleotides) or modified nucleotides, including tagged or labeled nucleotides. In some embodiments, a nucleic acid includes only naturally occurring nucleotides. In some embodiments, a nucleic acid includes only non-naturally occurring nucleotides. In some embodiments, a nucleic acid includes combinations of naturally occurring and non-naturally occurring nucleotides. In some embodiments, a nucleic acid is single-stranded. In some embodiments, a nucleic acid is double-stranded. In some embodiments, a nucleic acid has combinations of single and double stranded regions. The term nucleic acid also encompasses hybrid molecules that have mixtures of ribonucleotides, deoxyribonucleotides, nucleotide analogues (such a bridged nucleotides), and/or modified nucleotides, including tagged or labeled nucleotides. In some aspects, disruption of a nucleic acid may be advantageous to generate smaller nucleic acid fragments. In some embodiments, disruption is performed by any of the following: sonication (i.e., hydrodynamic shearing), acoustic shearing, needle shearing, French pressure cells, or enzymatic (e.g., restriction) digestion. As used herein, the term “promoter” refers to a region of a nucleic acid that initiates transcription of a nucleic acid template by a RNA polymerase.

As used herein, the term “oligonucleotide” refers to a short nucleic acid. In some embodiments, an oligonucleotide is 2 to 250 nucleotides in length, 2 to 100 nucleotides in length, 10 to 100 nucleotides in length, 10 to 50 nucleotides in length, or 10 to 30 nucleotides in length. In some embodiments, an oligonucleotide is up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 150, 200 or 250 nucleotides in length. In some embodiments, an oligonucleotide is single-stranded. In some embodiments, an oligonucleotide is double-stranded. In some embodiments, an oligonucleotide comprises a hybridization sequence that hybridizes with a target nucleic acid by forming complementary base pairs with at least a portion of target nucleic acid.

In some embodiments, an oligonucleotide has a 3′-end capable of priming an extension reaction. In some embodiments, a hybridization sequence sequence may be 6 to 50 nucleotides in length, 6 to 35 nucleotides in length, 6 to 20 nucleotides in length, 10 to 25 nucleotides in length. A oligonucleotide is capable of “hybridizing” with another nucleic acid, such as a cDNA, genomic DNA, or RNA, when a single stranded form of the nucleic acid molecule or hybridization sequence thereof can anneal to the other nucleic acid molecule under the appropriate conditions of temperature and solution ionic strength. Hybridization occurs when two nucleic acids contain sufficiently complementary sequences, and depending on the stringency of the hybridization, mismatches between bases are possible. In some embodiments, the appropriate stringency for hybridizing nucleic acids depends on the length of the nucleic acids and the degree of complementation, GC content, and other parameters. In some embodiments, the greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. The term “complementary” describes the relationship between nucleotide bases that are capable to hybridizing to one another. For example, with respect to DNA, adenosine is complementary to thymine and cytosine is complementary to guanine.

In some embodiments the GC content of an oligonucleotide is approximately 20%, 25%, 30%, 35%, 40%, 45%, 50%, 55%, 60%, 65%, 70%, 75%, 80% or more. In some embodiments the GC content of an oligonucleotide is in a range of 20% to 80%, 20% to 70%, 35% to 65%, 40% to 60%, or 45% to 55%. In some embodiments, the oligonucleotides contain multiple (e.g., 2-3, 2-4, 2-5 or more) guanine or cytosine nucleotides on the 3′ end (e.g., GC clamp).

In some embodiments, oligonucleotides disclosed herein contain one or more modified nucleotides. In some embodiments the 5′ and/or 3′ end of the oligonucleotide is modified. In some embodiments, one or more internal nucleotides are modified. The oligonucleotide can be modified at the base moiety, sugar moiety, or phosphate backbone, for example, to improve stability of the molecule, resistance to nuclease mediate degradation, its hybridization parameters, etc. In some embodiments, an oligonucleotide may comprise a modified base moiety which is selected from: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, and 2,6-diaminopurine. Further examples of modifications include methylation, incorporation of “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.) and combinations thereof. Furthermore, the oligonucleotides herein may also be modified in some embodiments with a label capable of providing a detectable signal, either directly or indirectly. Exemplary labels include radioisotopes, fluorescent molecules, biotin, and others. In some embodiments, oligonucleotides disclosed herein contain a 5′ biotin linker or other suitable linker. In some embodiments, an oligonucleotide contains a restriction digestion sequence such that cleavage with the appropriate restriction digestion enzyme results in removal of the linker. In other embodiments, the 5′ end of an oligonucleotide contains a nucleic acid sequence that is complementary to a nucleic acid bound to a bead or other support, e.g., a flow cell substrate.

In some embodiments, where multiple oligonucleotides are combined in a common reaction, the oligonucleotides are designed to minimize or prevent formation of homo or hetero multimers (e.g., homo or heterodimers).

In some embodiments, a oligonucleotide comprises a hybridization sequence that is complementary with a target sequence of a nucleic acid, in which the target sequence is within a predetermined distance from a junction between a known sequence of the nucleic acid and an adjacent sequence. In some embodiments, the junction is a junction between fragments in a genome assembly. In some embodiments, the junction is a breakpoint resulting in a fusion between two nucleic acids (e.g., a breakpoint resulting from a genomic rearrangement). In some embodiments, an end of the target sequence is within 10, 20, 30, 40, 50, 60, 70, 80, 90, 100, 200, 300 or more nucleotides of the junction between a known sequence of a nucleic acid and an adjacent sequence (e.g., an unknown sequence).

In some embodiments, use in preparative methods disclosed herein of target specific oligonucleotides having hybridization sequences that are complementary to different target sequences (e.g., target sequence #1 and target sequence #2) in the same direction or orientation on a template facilitates amplification of templates that may otherwise be difficult to target with opposing primers. In such embodiments, use of target specific oligonucleotides having hybridization sequences that are complementary to different target sequences in the same direction or orientation provides the specificity benefit of two hybridization sequences complementary with a known region of a template, without utilizing a target specific oligonucleotide having a hybridization sequence that is complementary to a target sequence in the opposite direction to get coverage over a targeted region. Thus, in some embodiments, use of oligonucleotides complementary to target sequences in the same direction or orientation facilitates tiling across long template regions in the common reaction.

In some embodiments, oligonucleotides (e.g., target oligonucleotides, oligonucleotide having different hybridization sequences) may also contain additional functional sequences. In some embodiments, additional sequences are incorporated into a nucleic acid through amplification of a target sequences using a oligonucleotide comprising at its 5′-end the additional sequences. In some embodiments, oligonucleotides containing a common sequence also contain additional sequences. In some embodiments, target-specific oligonucleotides also contain additional sequences. In some embodiments, the additional sequences comprise one or more of the following non-limiting examples: identifier sequences (e.g., barcode, index), sequencing primer hybridization sequences (e.g., Rd1), and adapter sequences. In some embodiments the adapter sequences are sequences used with a next generation sequencing system. In some embodiments, the adapter sequences are P5 (SEQ ID NO:62) and P7 (SEQ ID NO: 63) sequences for Illumina-based sequencing technology. In some embodiments, the adapter sequence are P1 (SEQ ID NO: 64) and A (SEQ ID NO: 65) compatible with Ion Torrent sequencing technology.

As used herein, a “barcode” or “index” sequence is a nucleotide sequence that serves as a source or location identifier for the nucleic acid. For example, a barcode or index sequence may serve to identify a patient from whom a nucleic acid template is obtained to be processed and sequenced. In some embodiments, a barcode or index sequences that is incorporated into a DNA fragment enables sequencing of multiple different samples on a single flow cell. In some embodiments, an index sequence can be used to orientate a sequence imager for purposes of detecting individual sequencing reactions. In some embodiments, a barcode or index sequence may be 2 to 25 nucleotides in length, 2 to 15 nucleotides in length, 2 to 10 nucleotides in length, 2 to 6 nucleotides in length.

As used herein, a “adapter” sequence refers to a sequence used to attach a nucleic acid (e.g., an amplified DNA product) to a next generation sequencing platform or other substrate for purposes of immobilizing the nucleic acid. In some embodiments, an adapter sequence contains a sequencing primer hybridization sequence. In some embodiments, an adapter sequence contains P5 (SEQ ID NO:62) and/or P7 (SEQ ID NO: 63) sequences for Illumina-based sequencing. In some embodiments, an adapter sequence contains a P1 (SEQ ID NO: 64) and/or A (SEQ ID NO: 65) sequence that is compatible with Ion Torrent sequencing technology. In some embodiments, an adapter sequence may be 4 to 50 nucleotides in length, 4 to 30 nucleotides in length, 4 to 20 nucleotides in length, 15 to 30 nucleotides in length.

As used herein, the term “amplification” or “amplifying” refers to a process of increasing the number of copies of a nucleic acid template. In some embodiments, amplification involve the use of one or more polymerases that synthesize nucleic acids from a template. In some embodiments, amplification is accomplished under isothermal conditions. In some embodiments, amplification is accomplished under conditions involving multiple thermal cycles, such as in a polymerase chain reaction. In some embodiments, amplification involves one or more template-dependent extensions that are primed by an oligonucleotide that hybridizes at its 3′-end to a template. In some embodiments, a template-dependent extension is carried out by a reverse transcriptase. In some embodiments, a template-dependent extension is carried out by a DNA polymerase. In some embodiments, the template-dependent extension is performed by a reverse transcriptase enzyme that also contains DNA polymerase activity. A template dependent extension reaction may be carried out using any appropriate nucleic acid as a template. In some embodiments, a template-dependent extension is carried out on a DNA template. In some embodiments, a template-dependent extension is carried out on a RNA template. In some embodiments, amplification involves one or more transcription reactions. In some embodiments, amplification involves one or more template-dependent extensions in combination with one or more transcription reactions. In some embodiments, amplification results in a linear increase in the number of copies of a nucleic acid template. In some embodiments, in a linear amplification, one or more copies of a nucleic acid are produced from a single set of one or more nucleic acid templates. In some embodiments, amplification results in an exponential increase in the number of copies of a nucleic acid template. In some embodiments, in an exponential amplification, newly formed copies of a nucleic acid serve as templates for the production of further copies of the template resulting in an exponentially amplified pool of nucleic acids.

As used herein, the term “template” refers to a double-stranded or single-stranded nucleic acid that serves a substrate for nucleic acid synthesis, e.g., for a template-dependent extension or a transcription reaction. In the case of a double-stranded DNA molecule, denaturation of at least a portion of its two strands may be performed prior to or in conjunction with nucleic acid synthesis. In some embodiments, a template is single stranded and denaturation is not needed prior to or in conjunction with nucleic acid synthesis. In some embodiments, when an oligonucleotide complementary to a portion of a nucleic acid template is hybridized via a hybridization sequence to a template, an appropriate polymerase may then synthesize a nucleic acid complementary to the template. In some embodiments, an RNA polymerase may synthesize from a promoter region a nucleic acid complementary to an antisense strand of the template. In some embodiments, a template is a nucleic acid having a length in the range of up to 10, 20, 30, 40, 50, 60, 70, 80, 90, 100, 200, 300, 400, 500, 1000, 2000, 3000 or more nucleotides.

As used herein, the term “template-dependent extension” refers to an process in which an oligonucleotide that is hybridized at its 3′-end, through a hybridization sequence, to a complementary sequence of a single-stranded nucleic acid template is enzymatically extended through sequential covalent bonding of complementary nucleotides to the 3′-end of the oligonucleotide forming a new nucleic acid complementary to the template. In some embodiments, a template-dependent extension results in a partially or completely double-stranded nucleic acid with the extension product hybridized to the template.

In some embodiments, an extension reaction involves an oligonucleotide that hybridizes to complementary regions of a template nucleic acid and functions to prime an extension reaction to generate a complementary DNA strand. In some embodiments, synthesis of a complementary DNA strand from a template may be performed by a DNA polymerase enzyme. In some embodiments, DNA Polymerase I is used under conditions in which the enzyme performs a template-dependent extension. Non-limiting examples of DNA polymerases that are also able to perform this function include Taq polymerase, Pheonix Taq polymerase, Phusion polymerase, T4 polymerase, T7 polymerase, Klenow fragment, Klenow exo-, phi29 polymerase, AMY reverse transcriptase, M-MuLV reverse transcriptase, HIV-1 reverse transcriptase, VeraSeq ULtra polymerase, and EnzScript. In some embodiments, the DNA polymerase is not a reverse transcriptase. In some embodiments, the DNA polymerase acts on a DNA template. In some embodiments, the DNA polymerase acts on an RNA template.

In some embodiments, an extension reaction involves reverse transcription performed on an RNA to produce a complementary DNA molecule (RNA-dependent DNA polymerase activity). In some embodiments, a reverse transcriptase from mouse molony murine leukemia virus (M-MLV) may be used. It should be appreciated that many other reverse transcriptases may be used, including, but not limited to, AMV reverse transcriptase, RSV reverse transcriptase, HIV-1 reverse transcriptase, HIV-2 reverse transcriptase or others disclosed herein.

As used herein, the term “extension product” refers to a nucleic acid complementary to a nucleic acid template and formed by a template-dependent extension. In some embodiments, the 3′-end of the hybridization sequence of an oligonucleotide that hybridizes with a nucleic acid template serves as a primer for a template-dependent extension that results in a new nucleic acid complementary to the nucleic acid template. An extension product may be a fully or partially complementary to the nucleic acid template from which it was produced.

In some embodiments, an extension product is produced using an oligonucleotide having a hybridization sequence that is complementary with a target nucleic acid and an additional sequence 5′ to the hybridization sequence that is non-complementary with the template and that is incorporated into the 5′ end of the extension product. Such additional sequences may comprise a tag, barcode, index, adapter or other sequences for purposes of incorporating desired features into an extension product. In some embodiments, a partially complementary extension product is produced when an extension reaction does not extend for the full length of the template.

In some embodiments, a partially complementary extension product is primed at an internal sequence of a template. In some embodiments, a partially complementary extension product has a 3′ region that is fully complementary with a template sequence and a 5′ region that is non-complementary, in which the non-complementary 5′ region is an additional sequence of the oligonucleotide that primed the extension reaction that produced the extension product.

As used herein, the term “isothermal reaction” refers to a reaction that involves one or more enzymes acting on a nucleic acid template to produce copies of the template or portions of the template under relatively uniform temperature conditions. In some embodiments, the isothermal reaction involves exponentially amplifying DNA and/or RNA molecules under relatively uniform temperature conditions in preparation for sequencing. In some embodiments, an isothermal reaction is performed under relatively uniform temperature conditions under steady state reaction conditions. In some embodiments, an isothermal reaction involves one or more rounds of amplification performed at a relatively uniform temperature. In some embodiments, isothermal reactions are performed in a range of 35° C. to 50° C., 38° C. to 42° C., 39° C. to 42° C. or 35° C. to 45° C. (e.g., about 41° C.). In some embodiments, isothermal reactions are performed at about 35° C., 36° C., 37° C., 38° C., 39° C., 40° C., 41° C., 42° C., 43° C., 44° C., 45° C., 46° C., 47° C., 48° C., 49° C., or 50° C.

As used herein, the term “polymerase” refers to an enzyme that synthesizes nucleic acids. The term encompasses DNA polymerases, RNA polymerases, and reverse transcriptases, among others. In some embodiments, a polymerase synthesizes a nucleic acid through a template-dependent extension that is primed by an oligonucleotide that hybridizes at its 3′-end to a template. In some embodiments, a polymerase synthesizes a nucleic acid through a transcription reaction. In some embodiments, a polymerase optimally synthesizes a nucleic acid under suitable buffer conditions and at a temperature in range of 35° C. to 80° C. or 35° C. to 75° C. or 35° C. to 70° C. or 35° C. to 70° C. or 35° C. to 65° C. or 35° C. to 60° C. or 35° C. to 55° C. or 35° C. to 50° C. or 35° C. to 45° C. or 40° C. to 70° C. or 50° C. to 60° C. or 55° C. to 65° C.

In some embodiments, a polymerase is a thermostable polymerase. In some embodiments the thermostable polymerase is thermophilic Eubacteria or Archaebacteria polymerase from, for example, Thermus aquaticus, T. thermophilus, T. bockianus, T flavus, T. rubber, Thermococcus litoralis, Pyroccocus furiousus, P. wosei, Pyrococcus spec., Thermatoga maritime, Thermoplasma acidophilus, or Sulfolobus spec.

In some embodiments, methods disclosed herein involve degradation of RNA. One non-limiting example of such an enzyme is RNase H that degrades the RNA strand of a RNA-DNA hybrid. Additional examples of ribonucleases include RNase A, RNase I, RNase III, and RNase L. In some embodiments, RNA is degraded using a non-enzymatic method. It is appreciated that there exist many methods and reagents to degrade RNA in a reaction, including but not limited to increasing pH of a solution, which may be accomplished by many methods including but not limited to the addition of NaOH, as well as increasing the temperature of the reaction. In some embodiments, the pH of the reaction is increased by addition of NaOH to a pH of 10.0.

In some embodiments, method disclosed herein involve removal or degradation of excess oligonucleotides and other nucleic acids in a reaction. In some embodiments, nucleic acids are degraded enzymatically. In some embodiments, E. coli Exonuclease I is used to degrade single stranded nucleic acids in the presence of buffers and conditions that allow for the enzyme to perform the indicated activity.

In some embodiments, method disclosed herein involve ligation of double stranded adapter to either end of a DNA fragment. In this context, ligation refers to the covalent phosphodiester bond between two nucleic acids. In some embodiments, the ligation will occur between two double stranded DNA molecules, for example the double stranded adapter and the double stranded DNA fragment, such that covalent phosphodiester bonds are formed between both strands of the DNA. In some embodiments, ligation activity is performed enzymatically by a ligase. Any of the following non-limiting list of enzymes with ligase activity may be used to perform this reaction: E. coli DNA ligase, T4 DNA ligase, Taw DNA ligase, T3 DNA ligase.

In some embodiments, methods disclosed herein involve joining a RNA polymerase promoter sequence with a nucleic acid. Addition of the RNA polymerase promoter allows for recognition by an RNA polymerase that will perform DNA-dependent RNA polymerase activity producing a complementary RNA strand. Example, of such promoters include, but are not limited to, T7, T3, and SP6 promoters.

In some embodiments, methods disclosed herein involve purification of nucleic acids from a reaction mixture in preparation for subsequent steps or analysis. In some embodiments a RNA purification is performed. In some embodiments, any appropriate purification method may be used. In some embodiments, AMPure kit that uses a bead-based solid-phase extraction may be used. In some embodiments, additional methods include but are not limited to other solid-phase extraction methods (e.g., column-based methods) and liquid-liquid extraction methods (e.g., phenol-chloroform).

In some embodiments, kits are provided that comprise various reagents necessary to perform the preparative and/or sequencing reactions and instructions of use according to the methods set forth herein. Any of the enzymes, oligonucleotides, nucleic acids, or reagents disclosed herein may be formulated in a kit in any combination. In some embodiments of the kits, at least one container is a reusable container. In some embodiments of the kits, at least one container is a single-use container. In some embodiments of the kits, at least one container is a tube, bottle or cartridge (e.g., a multi-well cartridge). In some embodiments, kits are configured such that one or more processes are performed at different locations in a cartridge. In some embodiments, a cartridge may be loaded with or contain dried down or lyophilized components for each set of steps or processes for preparing nucleic acids. In some embodiments, kits provided herein include a tube, e.g., a snap-top tube or a screw-top tube. In some embodiments of the kits, the bottle is a snap-top bottle or a screw-top bottle. In some embodiments, at least one container is a glass vial. In some embodiments, containers are housed together in a box or a package. In some embodiments, kits further comprise instructions for preparing nucleic acids (e.g., in accordance with one or more steps outlined in Examples 1-3). In some embodiments, the kits further comprise instructions for storing at least one container at a particular temperature (e.g., less than 0° C., room temperature, etc.). In some embodiments, the kits further comprise instructions for carrying out any of the methods disclosed herein using reagents provided in the kits.

While in some embodiments the kits disclosed herein are useful for research purposes, in other embodiments, the kits disclosed herein are useful for diagnostic purposes. Accordingly, in some embodiments, the kits contain one or more reagents or components useful in methods for diagnosing or aiding in diagnosing an individual as having a disorder, e.g., cancer.

Exemplary embodiments of the invention will be described in more detail by the following examples. These embodiments are exemplary of the invention, which one skilled in art will recognize is not limited to the exemplary embodiments.

### EXAMPLES

### Example 1

### An Isothermal Method Using Reverse Transcriptase with Tagged Randomers and Targeted Second Strand Oligonucleotides to Amplify 3′Fusion Events

### Amplification #1

As a first step towards amplifying a 3′fusion event with an unknown fusion partner, a first amplification reaction was performed using RNA obtained from a formalin fixed tissue sample.

The following reaction was assembled with reaction buffer at room temperature:
1 μL purified RNA (50 ng)
1 μL 3 μM V3-FGFR GSP1
1 μL 5 μM T7-N9 Randomer
2 μL dH₂O

The reaction was mixed gently, centrifuged for a few seconds and transferred to a thermal cycler where it was incubated at 65° C. for 2 minutes followed by 41° C. for 11.5 minutes.

In some embodiments, an enzyme mix is used. In some embodiments, the enzyme mix is lyophilized and reconstituted prior to use. In some embodiments, the enzyme mix comprises 2, 3 or more enzymes. In some embodiments, the enzyme mix further comprises a high molecular weight sugar mix (e.g., dextran). In some embodiments, the enzyme mix is an enzyme mixture for isothermal RNA amplification. In some embodiments, the enzyme mix comprises reverse transcriptase (e.g., avian myeloblastosis virus reverse transcriptase), RNase H and RNA polymerase (e.g., T7 RNA polymerase). In some embodiments, the enzyme mix comprises 4 to 10 units of reverse transcriptase, 0.01 to 0.1 units of RNase H, and 10 to 40 units of RNA polymerase.

During the oligonucleotide-RNA annealing reaction above, the enzyme mix was prepared. The diluent for the lyophilized enzyme mix was thawed, then 30 μL of cold diluent was added to the lyophilized enzyme mix and incubated for 6 minutes. Immediately following the annealing reaction above, 5 μL at of the enzyme mix was added to each of the annealed RNA-oligonucleotide mixtures and incubated at 41° C. for 45-90 minutes.

### RNA Purification Using RNA XP AMPure Beads

The following was added to the reaction above,
36 μL AMPure beads

The suspension was mixed well and incubated for 5 minutes at room temperature. A magnet was used 2-4 minutes to collect the beads and the solution appeared clear. The supernatant was discarded and the beads were washed three times with 200 μL 70% ethanol on the magnet. After the second wash, the beads were dried at room temperature for 10 minutes. Finally, the RNA was eluted by removing the tubes from the magnet and resuspending the beads in 15 μL 10 mM Tris-HCl pH 8.3 elution buffer included in the AMPure kit. The RNA-bead solution was placed on the magnet for 2 minutes and then the RNA solution was transferred to a fresh PCR tube, being sure to avoid transferring beads to the fresh tube.

### Amplification #2

The following reaction was assembled with reaction buffer at room temperature:
2 μL from Amplification reaction #1, above
1 μL 3 μM V3-FGFR GSP1
1 μL 5 μM T7-cRNA R2P
1 μL dH₂O

The reaction was mixed gently, centrifuged for a few seconds and transferred to a thermal cycler where it was incubated at 65° C. for 2 minutes followed by 41° C. for 11.5 minutes.

During the oligonucleotide-RNA annealing reaction above, the enzyme mix was prepared. The diluent for the lyophilized enzyme mix was thawed, then 30 μL of cold diluent was added to the lyophilized enzyme mix and incubated for 6 minutes. Immediately following the annealing reaction above, 5 μL of the enzyme mix was added to each of the annealed RNA-oligonucleotide mixtures and incubated at 41° C. for 45-90 minutes.

### RNA Purification Using RNA XP AMPure Beads

RNA purification was performed as above, eluting the RNA in 15 μL 10 mM Tris-HCl pH8.0 Elution Buffer.

### Amplification #3

The following reaction was assembled with reaction buffer at room temperature:
1 μL from Amplification reaction #2, above
1 μL 3 μM V3-FGFR GSP2
1 μL 5 μM T7-cRNA R2P
2 μL dH₂O

The reaction was mixed gently, centrifuged for a few seconds and transferred to a thermal cycler where it was incubated at 65° C. for 2 minutes followed by 41° C. for 11.5 minutes.

During the oligonucleotide-RNA annealing reaction above, the enzyme mix was prepared. The diluent for the lyophilized enzyme mix was thawed, then 30 μL of cold diluent was added to the lyophilized enzyme mix and incubated for 6 minutes. Immediately following the annealing reaction above, 5 μL of the enzyme mix was added to each of the annealed RNA-oligonucleotide mixtures and incubated at 41° C. for 45-90 minutes.

### RNA Purification Using RNA XP AMPure Beads

RNA purification was performed as above, this time eluting the RNA in 6 μL 10 mM Tris-HCl pH8.3 Elution Buffer.

### Amplification #4

The following reaction was assembled with reaction buffer at room temperature:
5 μL from Amplification #3, above
1 μL 10 μM P5
1 μL 10 μM P7

The reaction was mixed gently, centrifuged for a few seconds and transferred to a thermal cycler where it was incubated at 65° C. for 2 minutes followed by 41° C. for 11.5 minutes.

During the oligonucleotide-RNA annealing reaction above, the enzyme mix was prepared. The diluent for the lyophilized enzyme mix was thawed, then 30 μL of cold diluent was added to the lyophilized enzyme mix and incubated for 6 minutes. Immediately following the annealing reaction above, 5 μL of the enzyme mix was added to each of the annealed RNA-oligonucleotide mixtures and incubated at 41° C. for 45-90 minutes.

### DNA Purification Using DNA XP AMPure Heads

The following was added to the reaction above,
36 μL AMPure beads

The suspension was mixed well and incubated for 5 minutes at room temperature. A magnet was used 2-4 minutes to collect the beads and the solution appeared clear. The supernatant was discarded and the beads were washed three times with 200 μL 70% ethanol on the magnet. After the second wash, the beads were dried at room temperature for 5 minutes. Finally, the DNA was eluted by removing the tubes from the magnet and resuspending the beads in 15 μL 10 mM Tris-HCl pH 8.3 elution buffer included in the AMPure kit. The RNA-bead solution was placed on the magnet for 2 minutes. Then, the DNA solution was transferred to a fresh PCR tube, being sure to avoid transferring beads to the fresh tube. This final DNA product is then sequencing ready.

### Example 2

### An Isothermal Method Using Reverse Transcriptase with Tagged Randomers and Targeted Second Strand Oligonucleotides to Amplify 5′Fusion Events

### Amplification #1

The first step towards amplifying a genetic locus containing a 5′fusion event with an unknown fusion partner is to perform a reverse transcriptase event on the obtained sample RNA using a target/gene-specific primer(s).

The following reaction was assembled with reaction buffer at room temperature:
1 μL purified RNA (50 ng)
1 μL 3 μM V3-FGFR GSP1
1 μL 5 μM T7-N9 Randomer
2 μL dH₂O

The reaction was mixed gently, centrifuged for a few seconds and transferred to a thermal cycler where it was incubated at 65° C. for 2 minutes followed by 41° C. for 11.5 minutes.

During the oligonucleotide-RNA annealing reaction above, the enzyme mix was prepared. The diluent for the lyophilized enzyme mix was thawed, then 30 μL of cold diluent was added to the lyophilized enzyme mix and incubated for 6 minutes. Immediately following the annealing reaction above, 5 μL of the enzyme mix was added to each of the annealed RNA-oligonucleotide mixtures and incubated at 41° C. for 45-90 minutes.

### RNA Purification Using RNA XP AMPure Beads

The following was added to the reaction above,
36 μL AMPure beads

The suspension was mixed well and incubated for 5 minutes at room temperature. A magnet was used 2-4 minutes to collect the beads and the solution appeared clear. The supernatant was discarded and the beads were washed three times with 200 μL 70% ethanol on the magnet. After the second wash, the beads were dried at room temperature for 10 minutes. Finally, the RNA was eluted by removing the tubes from the magnet and resuspending the beads in 15 μL 10 mM Tris-HCl pH 8.3 elution buffer included in the AMPure kit. The RNA-bead solution was placed on the magnet for 2 minutes and then the RNA solution was transferred to a fresh PCR tube, being sure to avoid transferring beads to the fresh tube.

### Amplification #2

The following reaction was assembled with reaction buffer at room temperature:
2 μL from Amplification #1, above
1 μL 3 μM V3-FGFR GSP1
1 μL 5 μM T7-cRNA R2P
1 μL dH₂O

The reaction was mixed gently, centrifuged for a few seconds and transferred to a thermal cycler where it was incubated at 65° C. for 2 minutes followed by 41° C. for 11.5 minutes.

During the oligonucleotide-RNA annealing reaction above, the enzyme mix was prepared. The diluent for the lyophilized enzyme mix was thawed, then 30 μL of cold diluent was added to the lyophilized enzyme mix and incubated for 6 minutes. Immediately following the annealing reaction above, 5 μL of the enzyme mix was added to each of the annealed RNA-oligonucleotide mixtures and incubated at 41° C. for 45-90 minutes.

### RNA Purification Using RNA XP AMPure Beads

RNA purification was performed as above, eluting the RNA in 15 μL 10 mM Tris-HCl pH8.0 Elution Buffer.

### Amplification #3

The following reaction was assembled with reaction buffer at room temperature:
1 μL from Amplification #2, above
1 μL 3 μM V3-FGFR GSP2
1 μL 5 μM T7-cRNA R2P
2 μL dH₂O

The reaction was mixed gently, centrifuged for a few seconds and transferred to a thermal cycler where it was incubated at 65° C. for 2 minutes followed by 41° C. for 11.5 minutes.

During the oligonucleotide-RNA annealing reaction above, the enzyme mix was prepared. The diluent for the lyophilized enzyme mix was thawed, then 30 μL, of cold diluent was added to the lyophilized enzyme mix and incubated for 6 minutes. Immediately following the annealing reaction above, 5 μL of the enzyme mix was added to each of the annealed RNA-oligonucleotide mixtures and incubated at 41° C. for 45-90 minutes.

### RNA Purification Using RNA XP AMPure Beads

RNA purification was performed as above, this time eluting the RNA in 6 μL 10 mM Tris-HCl pH8.3 Elution Buffer.

### Amplification #4

The following reaction was assembled with reaction buffer at room temperature:
5 μL from Amplification #3, above
1 μL 10 μM P5
1 μL 10 μM P7

The reaction was mixed gently, centrifuged for a few seconds and transferred to a thermal cycler where it was incubated at 65° C. for 2 minutes followed by 41° C. for 11.5 minutes.

During the oligonucleotide-RNA annealing reaction above, the enzyme mix was prepared. The diluent for the lyophilized enzyme mix was thawed, then 30 μL of cold diluent was added to the lyophilized enzyme mix and incubated for 6 minutes. Immediately following the annealing reaction above, 5 μL of the enzyme mix was added to each of the annealed RNA-oligonucleotide mixtures and incubated at 41° C. for 45-90 minutes.

### DNA Purification Using DNA XP AMPure Beads

The following was added to the reaction above,
36 μL AMPure beads

The suspension was mixed well and incubated for 5 minutes at room temperature. A magnet was used 2-4 minutes to collect the beads and the solution appeared clear. The supernatant was discarded and the beads were washed three times with 200 μL 70% ethanol on the magnet. After the second wash, the beads were dried at room temperature for 5 minutes. Finally, the DNA was eluted by removing the tubes from the magnet and resuspending the beads in 15 μL 10 mM Tris-HCl pH 8.3 elution buffer included in the AMPure kit. The RNA-bead solution was placed on the magnet for 2 minutes. Then, the DNA solution was transferred to a fresh PCR tube, being sure to avoid transferring beads to the fresh tube. This final DNA product is then sequencing ready.

### Example 3

### An Isothermal Method to Exponentially Amplify Target Sequences Beginning with Genomic DNA

To prepare target regions of double-stranded genomic DNA for analysis, the DNA was first fragmented to between 100-600 bp in size.

In order to repair the ends of the fragments, then phosphorylate and adenylate opposing ends, the following reaction was prepared:
10 μL fragmented DNA (50-250 ng)
4 μL end-repair buffer
1 μL end-repair mix
1 μL Taq polymerase (A-tailing)
1 μL 2 mM dNTPs

The reaction was mixed gently and incubated in a thermal cycler at 12° C. for 15 minutes, 37° C. for 15 minutes, then 72° C. for 15 minutes, followed by 4° C. until proceeding with the next steps.

During the reaction above, the oligonucleotide adapter sequences containing 5′ RNA polymerase promoter sequences were annealed by mixing equal volumes of each oligonucleotide, heating to 95° C. and allowing to cool to room temperature.

The following ligation reaction was assembled at room temperature:
40 μL DNA prepared above
1 μL MiSeq Index 2 Adapter
4.9 μL Ligation Buffer
2 μL DNA Ligase

The reaction is then allowed to proceed at 16° C. for 30 minutes, then 22° C. for 30 minutes, followed by 4° C.

Purify with Ampure XP beads at 1.8× reaction volume.

### Amplification #1

The following reaction was assembled with reaction buffer at room temperature:
2 μL purified DNA from the ligation reaction, above
1 μL primer T7-R2P cRNA 5 μM
1 μL GSP1 3 μM
1 μL dH₂O

The reaction was mixed gently, centrifuged for a few seconds and transferred to a thermal cycler where it was incubate at 65° C. for 2 minutes followed by 41° C. for 11.5 minutes.

During the oligonucleotide-DNA annealing reaction above, the enzyme mix was prepared. The diluent for the lyophilized enzyme mix was thawed, then 30 μL of cold diluent was added to the lyophilized enzyme mix and incubated for 6 minutes. Immediately following the annealing reaction above, 5 μL of the enzyme mix was added to each of the annealed RNA-oligonucleotide mixtures and incubated at 41° C. for 45-90 minutes.

### RNA Purification Using RNA XP AMPure Beads

The following was added to the reaction above,
36 μL AMPure beads

The suspension was mixed well and incubated for 5 minutes at room temperature. A magnet was used 2-4 minutes to collect the beads and the solution appeared clear. The supernatant was discarded and the beads were washed three times with 200 μL 70% ethanol on the magnet. After the second wash, the beads were dried at room temperature for 10 minutes. Finally, the RNA was eluted by removing the tubes from the magnet and resuspending the beads in 15 μL 10 mM Tris-HCl pH 8.3 elution buffer included in the AMPure kit. The RNA-bead solution was placed on the magnet for 2 minutes and then the RNA solution was transferred to a fresh PCR tube, being sure to avoid transferring beads to the fresh tube.

### Amplification #2

The following reaction was assembled with reaction buffer at room temperature:
2 μL RNA from Amplification #1, above
1 μL T7-R2P-cRNA 5 uM
1 μL GSP2 3 uM
1 μL dH₂O

The reaction was mixed gently, centrifuged for a few seconds and transferred to a thermal cycler where it was incubate at 65° C. for 2 minutes followed by 41° C. for 11.5 minutes.

During the oligonucleotide-DNA annealing reaction above, the enzyme mix was prepared. The diluent for the lyophilized enzyme mix was thawed, then 30 μL of cold diluent was added to the lyophilized enzyme mix and incubated for 6 minutes, Immediately following the annealing reaction above, 5 μL of the enzyme mix was added to each of the annealed RNA-oligonucleotide mixtures and incubated at 41° C. for 45-90 minutes.

### Amplification #4

The following reaction was assembled with reaction buffer at room temperature:
5 μL from Amplification #3, above
1 μL 10 μM P5
1 μL 10 μM P7

The reaction was mixed gently, centrifuged for a few seconds and transferred to a thermal cycler where it was incubated at 65° C. for 2 minutes followed by 41° C. for 11.5 minutes.

During the oligonucleotide-RNA annealing reaction above, the enzyme mix was prepared. The diluent for the lyophilized enzyme mix was thawed, then 30 μL of cold diluent was added to the lyophilized enzyme mix and incubated for 6 minutes. Immediately following the annealing reaction above, 5 μL of the enzyme mix was added to each of the annealed RNA-oligonucleotide mixtures and incubated at 41° C. for 45-90 minutes.

DNA purification using DNA XP AMPure beads

The following was added to the reaction above,
36 μL AMPure beads

The suspension was mixed well and incubated for 5 minutes at room temperature. A magnet was used 2-4 minutes to collect the beads and the solution appeared clear. The supernatant was discarded and the beads were washed three times with 200 μL 70% ethanol on the magnet. After the second wash, the beads were dried at room temperature for 10 minutes. Finally, the DNA was eluted by removing the tubes from the magnet and resuspending the beads in 15 μL 10 mM Tris-HCl pH 8.3 elution buffer included in the AMPure kit. The DNA-bead solution was placed on the magnet for 2 minutes and then the DNA solution was transferred to a fresh PCR tube, being sure to avoid transferring heads to the fresh tube. This final DNA product is then sequencing ready.

### Example 4

### An Isothermal Method Using Reverse Transcriptase with Tagged Randomers and Random Second Strand Oligonucleotides to Amplify Unknown 5′ and/or 3′ Fusion Events

### Amplification #1

As a first step towards amplifying a 3′fusion event with an unknown fusion partner, a first amplification reaction was performed using RNA obtained from a formalin fixed tissue sample.

The following reaction was assembled with reaction buffer at room temperature:
1 μL purified RNA (50 ng)
1 μL 5 μM T7-N9 Randomer
3 μL dH₂O

The reaction was mixed gently, centrifuged for a few seconds and transferred to a thermal cycler where it was incubated at 65° C. for 2 minutes followed by 41° C. for 11.5 minutes.

During the oligonucleotide-RNA annealing reaction above, the enzyme mix was prepared. The diluent for the lyophilized enzyme mix was thawed, then 30 μL of cold diluent was added to the lyophilized enzyme mix and incubated for 6 minutes. Immediately following the annealing reaction above, 5 μL of the enzyme mix was added to each of the annealed RNA-oligonucleotide mixtures and incubated at 41° C. for 45-90 minutes.

### RNA Purification Using RNA XP AMPure Heads

The following was added to the reaction above,
36 μL AMPure beads

The suspension was mixed well and incubated for 5 minutes at room temperature. A magnet was used 2-4 minutes to collect the beads and the solution appeared clear. The supernatant was discarded and the beads were washed three times with 200 μL 70% ethanol on the magnet. After the second wash, the beads were dried at room temperature for 10 minutes. Finally, the RNA was eluted by removing the tubes from the magnet and resuspending the beads in 15 μL 10 mM Tris-HCl pH 8.3 elution buffer included in the AMPure kit. The RNA-bead solution was placed on the magnet for 2 minutes and then the RNA solution was transferred to a fresh PCR tube, being sure to avoid transferring beads to the fresh tube.

### Amplification #2

The following reaction was assembled with reaction buffer at room temperature:
2 μL from Amplification reaction #1, above
1 μL 3 μM GSP1 (5′ or 3′ directional primers)
1 μL 5 μM R2P
1 μL dH₂O

The reaction was mixed gently, centrifuged for a few seconds and transferred to a thermal cycler where it was incubated at 65° C. for 2 minutes followed by 41° C. for 11.5 minutes.

During the oligonucleotide-RNA annealing reaction above, the enzyme mix was prepared. The diluent for the lyophilized enzyme mix was thawed, then 30 μL of cold diluent was added to the lyophilized enzyme mix and incubated for 6 minutes. Immediately following the annealing reaction above, 5 μL of the enzyme mix was added to each of the annealed RNA-oligonucleotide mixtures and incubated at 41° C. for 45-90 minutes.

### RATA Purification Using RNA XP AMPure Heads

RNA purification was performed as above, eluting the RNA in 15 μL 10 mM Tris-HCl pH8.0 Elution Buffer,

### Amplification #3

The following reaction was assembled with reaction buffer at room temperature:
5 μL from Amplification #2, above
1 μL 10 μM P5
1 μL 10 μM P7

The reaction was mixed gently, centrifuged for a few seconds and transferred to a thermal cycler where it was incubated at 65° C. for 2 minutes followed by 41° C. for 11.5 minutes.

During the oligonucleotide-RNA annealing reaction above, the enzyme mix was prepared. The diluent for the lyophilized enzyme mix was thawed, then 30 μL of cold diluent was added to the lyophilized enzyme mix and incubated for 6 minutes. Immediately following the annealing reaction above, 5 μL of the enzyme mix was added to each of the annealed RNA-oligonucleotide mixtures and incubated at 41° C. for 45-90 minutes.

### DNA Purification Using DNA XP AMPure Heads

The following was added to the reaction above,
36 μL AMPure beads

The suspension was mixed well and incubated for 5 minutes at room temperature. A magnet was used 2-4 minutes to collect the beads and the solution appeared clear. The supernatant was discarded and the beads were washed three times with 200 μL 70% ethanol on the magnet. After the second wash, the beads were dried at room temperature for 5 minutes. Finally, the DNA was eluted by removing the tubes from the magnet and resuspending the beads in 15 μL 10 mM Tris-HCl pH 8.3 elution buffer included in the AMPure kit. The RNA-bead solution was placed on the magnet for 2 minutes. Then, the DNA solution was transferred to a fresh PCR tube, being sure to avoid transferring beads to the fresh tube. This final DNA product is then sequencing ready.

### Example 5

### Oligonucleotides

The following table provides an exemplary oligonucleotide sequences for amplification of nucleic acids in preparation for sequencing analysis, e.g., as described in Examples 1-3.

T7 randomer RT random oligonucleotides

| SEQ | | |
|---|---|---|
| ID | Name | Sequence |
| 1 | T7-N6 | GAAATTAATACGACTCACTATAGGGAAGAC |
| | | GTGTGCTCTTCCGATCTNNNNNN |
| 2 | T7-N9 | GAAATTAATACGACTCACTATAGGGAAGAC |
| | | GTGTGCTCTTCCGATCTNNNNNNNNN |
| 3 | T7-N15 | GAAATTAATACGACTCACTATAGGGAAGAC |
| | | GTGTGCTCTTCCGATCTNNNNNNNNNNNNN |
| | | NN |
| 4 | T7-cRNA-R2P | GAAATTAATACGACTCACTATAGGGAAGAC |
| | | GTGTGCTCTTCCGATCT |

3′ FGFR GSP1 oligonucleotides

| | | |
|---|---|---|
| 5 | FGFR_007_19 | GGATCTCGACGCTCTCCCTCAACCCTGCTT |
| | 3 GSP1 | GCAGGAT |
| 6 | FGFR1_008_14 | GGATCTCGACGCTCTCCCTCCTCCATCTCT |
| | 7 GSP 1 | TTGTCGGTGGT |
| 7 | FGFR1_009_20 | GGATCTCGACGCTCTCCCTATGAGGAAGGC |
| | 5 GSP1 | CCCTGTGC |
| 8 | FGFR1_010_14 | GGATCTCGACGCTCTCCCTCCCCAGAGTTC |
| | 8 GSP1 | ATGGATGCACT |
| 9 | FGFR1_011_12 | GGATCTCGACGCTCTCCCTAAAGCAGCCCT |
| | 4 GSP1 | CTCCCAGG |
| 10 | FGFR1_012_11 | GGATCTCGACGCTCTCCCTATTTCTGAGAT |
| | 3 GSP1 | CAGGTCTGACAAG |
| 11 | FGFR1_016_14 | GGATCTCGACGCTCTCCCTTGCTGAAGGAG |
| | 0 GSP1 | GGTCACCG |
| 12 | FGFR1_017_10 | GGATCTCGACGCTCTCCCTTTCAAGCAGCT |
| | 8 GSP1 | GGTGGAAGAC |
| 13 | FGFR1_018_17 | GGATCTCGACGCTCTCCCTCAGCCCAGCTT |
| | 8 GSP1 | GCCAATGGC |
| 14 | FGFR3_07_19 | GGATCTCGACGCTCTCCCTGTCTGGTTGGC |
| | 3 GSP1 | CGGCAGC |
| 15 | FGFR3_008_14 | GGATCTCGACGCTCTCCCTGACGTTGTGCA |
| | 7 GSP1 | AGGAGAGAACCT |
| 16 | FGFR3_009_19 | GGATCTCGACGCTCTCCCTCGCCTCGTCAG |
| | 3 GSP1 | CCTCCAC |
| 17 | FGFR3_010_14 | GGATCTCGACGCTCTCCCTACCAGTGGTGT |
| | 8 GSP1 | GTTGGAGCT |
| 18 | FGFR3_011_12 | GGATCTCGACGCTCTCCCTCGAAGCAGCCC |
| | 4 GSP1 | TCCCCAA |
| 19 | FGFR3_012_11 | GGATCTCGACGCTCTCCCTACCAGGTCCGA |
| | 3 GSP1 | CAGGTCC |
| 20 | FGFR3_016_14 | GGATCTCGACGCTCTCCCTCATGGACAAGC |
| | 0 GSP1 | CCGCCAA |
| 21 | FGFR3_017_10 | GGATCTCGACGCTCTCCCTGGAGGACCTGG |
| | 8 GSP1 | ACCGTGTC |
| 22 | FGFR3_018_14 | GGATCTCGACGCTCTCCCTCCTCAGGGGAC |
| | 8 GSP1 | GACTCCG |

3′ GSP2 FGFR RdI oligonucleotides

| | | |
|---|---|---|
| 23 | FGFR1_007_19 | ACACTCTTTCCCTACACGACGCTCTTCCGA |
| | 3-GSP2 | TCTCTTGCAGGATGGGCCGGTGA |
| 24 | FGFR1_008_14 | ACACTCTTTCCCTACACGACGCTCTTCCGA |
| | 7-GSP2 R1P | TCTCATCTCTTTGTCGGTGGTATTAACTCC |
| | | A |
| 25 | FGFR1_009_20 | ACACTCTTTCCCTACACGACGCTCTTCCGA |
| | 5-GSP2 R1P | TCTACAGGGGCGAGGTCATCAC |
| 26 | FGFR1_010_14 | ACACTCTTTCCCTACACGACGCTCTTCCGA |
| | 8-GSP2 R1P | TCTTGGATGCACTGGAGTCAGCA |
| 27 | FGFR1_011_12 | ACACTCTTTCCCTACACGACGCTCTTCCGA |
| | 4-GSP2 R1P | TCTCTCTCCCAGGGGTTTGCCTAA |
| 28 | FGFR1_012_11 | ACACTCTTTCCCTACACGACGCTCTTCCGA |
| | 3-GSP2 R1P | TCTGAGATCAGGTCTGACAAGTCTTTCTCT |
| | | G |
| 29 | FGFR1_012_11 | ACACTCTTTCCCTACACGACGCTCTTCCGA |
| | 3-GSP2 R1P | TCTGAGATCAGGTCTGACAAGTCTTTCTCT |
| | | G |
| 30 | FGFR1_016_14 | ACACTCTTTCCCTACACGACGCTCTTCCGA |
| | 0-GSP2 R1P | TCTGAGGGTCACCGCATGGACAAG |
| 31 | FGFR1_017_10 | ACACTCTTTCCCTACACGACGCTCTTCCGA |
| | 8-GSP2 R1P | TCTCATCGTGGCCTTGACCTCCA |
| 32 | FGFR1_018_17 | ACACTCTTTCCCTACACGACGCTCTTCCGA |
| | 8-GSP2 R1P | TCTGCCAATGGCGGACTCAAACG |
| 33 | FGFR3_007_19 | ACACTCTTTCCCTACACGACGCTCTTCCGA |
| | 3-GSP2 R1P | TCTCTGCAGGATGGGCCGGTG |
| 34 | FGFR3_008_14 | ACACTCTTTCCCTACACGACGCTCTTCCGA |
| | 7-GSP2 R1P | TCTAGCTCCTTGTCGGTGGTGTTAG |
| 35 | FGFR3_009_19 | ACACTCTTTCCCTACACGACGCTCTTCCGA |
| | 3-GSP2 R1P | TCTCGTCAGCCTCCACCAGCT |
| 36 | FGFR3_010_14 | ACACTCTTTCCCTACACGACGCTCTTCCGA |
| | 8-GSP2 R1P | TCTCCAGTGGTGTGTTGGAGCTCAT |
| 37 | FGFR3_011_12 | ACACTCTTTCCCTACACGACGCTCTTCCGA |
| | 4-GSP2 R1P | TCTCCCAAGGGGCTTGCCCAG |
| 38 | FGFR3_012_11 | ACACTCTTTCCCTACACGACGCTCTTCCGA |
| | 3-GSP2 R1P | TCTCCGACAGGTCCTTGTCAGTGG |
| 39 | FGFR3_016_14 | ACACTCTTTCCCTACACGACGCTCTTCCGA |
| | 0-GSP2 R1P | TCTCCCGCCAACTGCACACAC |
| 40 | FGFR3_017_10 | ACACTCTTTCCCTACACGACGCTCTTCCGA |
| | 8-GSP2 R1P | TCTGTGTCCTTACCGTGACGTCCA |
| 41 | FGFR3_018_14 | ACACTCTTTCCCTACACGACGCTCTTCCGA |
| | 8-GSP2 R1P | TCTCGACTCCGTGTTTGCCCAC |

P5 index tag oligonucleotide

| SEQ | | | Index to |
|---|---|---|---|
| ID | Name | Sequence | Machine |
| 42 | P5_1_v3 | AATGATACGGCGACCACCGA | TAGATCGC |
| | | GATCTACACTAGATCGCACA | |
| | | CTCTTTCCCTACACGACGCT | |
| | | CTTC | |
| 43 | P5_2_v3 | AATGATACGGCGACCACCGA | CTCTCTAT |
| | | GATCTACACCTCTCTATACA | |
| | | CTCTTTCCCTACACGACGCT | |
| | | CTTC | |
| 44 | P5_3_v3 | AATGATACGGCGACCACCGA | TATCCTCT |
| | | GATCTACACTATCCTCTACA | |
| | | CTCTTTCCCTACACGACGCT | |
| | | CTTC | |
| 45 | P5_4_v3 | AATGATACGGCGACCACCGA | AGAGTAGA |
| | | GATCTACACAGAGTAGAACA | |
| | | CTCTTTCCCTACACGACGCT | |
| | | CTTC | |
| 46 | P5_5_v3 | AATGATACGGCGACCACCGA | GTAAGGAG |
| | | GATCTACACGTAAGGAGACA | |
| | | CTCTTTCCCTACACGACGCT | |
| | | CTTC | |
| 47 | P5_6_v3 | AATGATACGGCGACCACCGA | ACTGCATA |
| | | GATCTACACACTGCATAACA | |
| | | CTCTTTCCCTACACGACGCT | |
| | | CTTC | |
| 48 | P5_7_v3 | AATGATACGGCGACCACCGA | AAGGAGTA |
| | | GATCTACACAAGGAGTAACA | |
| | | CTCTTTCCCTACACGACGCT | |
| | | CTTC | |
| 49 | P5_8_v3 | AATGATACGGCGACCACCGA | CTAAGCCT |
| | | GATCTACACCTAAGCCTACA | |
| | | CTCTTTCCCTACACGACGCT | |
| | | CTTC | |

P7 index tag oligonucleotide

| | | | |
|---|---|---|---|
| 50 | P7_1_v3 | CAAGCAGAAGACGGCATACG | TAAGGCGA |
| | | AGATTCGCCTTAGTGACTGG | |
| | | AGTTCAGACGTGTGCTCTTC | |
| | | CGATCT | |
| 51 | P_72_v3 | CAAGCAGAAGACGGCATACG | CGTACTAG |
| | | AGATCTAGTACGGTGACTGG | |
| | | AGTTCAGACGTGTGCTCTTC | |
| | | CGATCT | |
| 52 | P7_3_v3 | CAAGCAGAAGACGGCATACG | AGGCAGAA |
| | | AGATTTCTCTCCTGTGACTG | |
| | | GAGTTCAGACGTGTGCTCTT | |
| | | CCGATCT | |
| 53 | P7_4_v3 | CAAGCAGAAGACGGCATACG | TCCTGAGC |
| | | AGATGCTCAGGAGTGACTGG | |
| | | AGTTCAGACGTGTGCTCTTC | |
| | | CGATCT | |
| 54 | P7_5_v3 | CAAGCAGAAGACGGCATACG | GGACTCCT |
| | | AGATAGGAGTCCGTGACTGG | |
| | | AGTTCAGACGTGTGCTCTTC | |
| | | CGATCT | |
| 55 | P7_6_v3 | CAAGCAGAAGACGGCATACG | TAGGCATG |
| | | AGATCATGCCTAGTGACTGG | |
| | | AGTTCAGACGTGTGCTCTTC | |
| | | CGATCT | |
| 56 | P7_7_v3 | CAAGCAGAAGACGGCATACG | CTCTCTAC |
| | | AGATGTAGAGAGGTGACTGG | |
| | | AGTTCAGACGTGTGCTCTTC | |
| | | CGATCT | |
| 57 | P7_8_v3 | CAAGCAGAAGACGGCATACG | CAGAGAGG |
| | | AGATCCTCTCTGGTGACTGG | |
| | | AGTTCAGACGTGTGCTCTTC | |
| | | CGATCT | |
| 58 | P7_9_v3 | CAAGCAGAAGACGGCATACG | GCTACGCT |
| | | AGATAGCGTAGCGTGACTGG | |
| | | AGTTCAGACGTGTGCTCTTC | |
| | | CGATCT | |
| 59 | P7_10_v3 | CAAGCAGAAGACGGCATACG | CGAGGCTG |
| | | AGATCAGCCTCGGTGACTGG | |
| | | AGTTCAGACGTGTGCTCTTC | |
| | | CGATCT | |
| 60 | P7_11_v3 | CAAGCAGAAGACGGCATACG | AAGAGGCA |
| | | AGATTGCCTCTTGTGACTGG | |
| | | AGTTCAGACGTGTGCTCTTC | |
| | | CGATCT | |
| 61 | P7_12_v3 | CAAGCAGAAGACGGCATACG | GTAGAGGA |
| | | AGATTCCTCTACGTGACTGG | |
| | | AGTTCAGACGTGTGCTCTTC | |
| | | CGATCT | |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of′ a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of,” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first.” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of preparing a nucleic acid for analysis, the method comprising:
(a) producing a synthetic RNA from a nucleic acid template;
(b) exponentially amplifying the synthetic RNA in an isothermal reaction; and
(c) generating a cDNA from the exponentially amplified synthetic RNA, wherein the cDNA comprises at least one non-target sequence.

2. A method of determining a sequence of a nucleic acid template, the method comprising:
(a) producing a synthetic RNA from a nucleic acid template;
(b) exponentially amplifying the synthetic RNA in an isothermal reaction;
(c) generating a cDNA from the exponentially amplified synthetic RNA; and
(d) sequencing the cDNA.

3. The method of any one of claim 1, wherein the isothermal reaction comprises two or more cycles of template-dependent extension and RNA polymerase transcription.

4. The method of claim 3, wherein at least one template-dependent extension in each cycle is a reverse transcription.

5-6. (canceled)

7. The method of claim 1, wherein the isothermal reaction comprises a template-dependent extension that synthesizes a first DNA strand that is complementary to the synthetic RNA, resulting in formation of a RNA-DNA hybrid between the first DNA strand and the synthetic RNA.

8. The method of claim 7, wherein the isothermal reaction further comprises degradation of the synthetic RNA portion of the RNA-DNA hybrid.

9. The method of claim 8, wherein the degradation is enzymatically mediated degradation.

10. The method of claim 9, wherein the degradation is mediated by RNAse H.

11. The method of claim 7, wherein the isothermal reaction further comprises a template-dependent extension that synthesizes a second DNA strand that is complementary to the first DNA, resulting in formation of a double-stranded DNA comprising the first and second DNA strands.

12. The method of claim 11, wherein the isothermal reaction further comprises an RNA polymerase mediated transcription reaction that transcribes synthetic RNAs from the double-stranded DNA.

13. The method of claim 2, wherein step (b) is repeated.

14. The method of claim 13, wherein the amplified synthetic RNA is purified after each consecutive round of step (b), and the purified synthetic RNA is used as starting material for the subsequent round(s) of step (b).

15. The method of claim 14, wherein at least two of the isothermal reactions of repeated step (b) comprise template-dependent extensions that are primed by oligonucleotides having hybridization sequences that are complementary with nested sequences of the template synthetic RNA or first DNA strand.

16. The method of claim 14, wherein at least two of the isothermal reactions of repeated step (b) comprise template-dependent extensions that are primed by oligonucleotides having hybridization sequences that are complementary with the template synthetic RNA or first DNA strand and additional non-complementary sequences.

17. The method of claim 16, wherein the additional non-complementary sequences comprises one or more of a barcode sequence, an index sequence, or an adapter sequence.

18. The method of claim 2 further comprising producing the nucleic acid template by performing at least one extension reaction using a oligonucleotide that comprises a target-specific hybridization sequence; and performing at least one extension reaction using a plurality of different oligonucleotides that share a common sequence that is 5′ to different hybridization sequences.

19. The method of claim 2, wherein the nucleic acid template comprises a target region and an adjacent region.

20. The method of claim 19, wherein the target-specific hybridization sequence is complementary with the target region and wherein at least one of the different hybridization sequences is complementary with the adjacent region.

21. The method of claim 19, wherein the target region comprises a sequence of a first gene and the adjacent region comprises a sequence of a second gene.

22. (canceled)

23. The method of claim 2, wherein the nucleic acid template is a double-stranded DNA comprising a promoter, wherein the synthetic RNA is enzymatically produced through an RNA polymerase that specifically binds to the promoter and transcribes DNA downstream of the promoter.

24. (canceled)

25. The method of claim 2, wherein the synthetic RNA is transcribed from an intermediate double-stranded DNA produced from the nucleic acid template, wherein the nucleic acid template is an isolated RNA.

26. (canceled)

27. The method of claim 26, wherein the mRNA is fusion mRNA encoded from a chromosomal segment that comprises a genetic rearrangement.

28. The method of claim 27, wherein the nucleic acid template is a chromosomal segment that comprises a portion of a genetic rearrangement.

29. (canceled)

30. The method of claim 2, wherein the cDNA contains a non-template sequence that serves as a hybridization site for a sequencing primer that primes the sequencing reaction.

31. The method of claim 2, wherein the cDNA is sequenced in a multiplex reaction that includes different nucleic acids originating from different sources.

32. The method of claim 31, wherein the different sources are different subjects from which the nucleic acid templates were obtained.

33. The method of claim 32, wherein the different sources are different tissues from which the nucleic acid templates were obtained.

34. A method for sequencing a nucleic acid, the method comprising,
producing a synthetic RNA from a nucleic acid template that comprises a target region and an adjacent region;
producing a double-stranded nucleic acid that comprises a first strand synthesized by a template-dependent extension using the synthetic RNA as a template and a second strand synthesized by a template-dependent extension using the first strand as a template, wherein the double-stranded nucleic acid is representative of the target region and the adjacent region of the nucleic acid template; and
performing a sequencing reaction using the double-stranded nucleic acid to determine a nucleotide sequence of the target region and the adjacent region.

35-42. (canceled)

43. A kit for use in the method of claim 34, the kit comprising
a container housing a lyophilized composition that comprises at least one oligonucleotide comprising a hybridization sequence and RNA polymerase promoter sequence; a reverse transcriptase; a DNA polymerase; and an RNA polymerase.

44-49. (canceled)
